(19) United States
(12) Patent Application Publication
(10) Pub. No.: US 2011/0243859 A1
(43) Pub. Date: Oct. 6, 2011

(54) ENCAPSULATING SYSTEM FOR CEST IMAGING WITH CHELATE Q GREATER THAN OR EQUAL TO 2

(75) Inventor: Marc Port, Deuil La Barre (FR)

(73) Assignee: GUERBET, Villepinte (FR)

(21) Appl. No.: 13/133,902

(22) PCT Filed: Dec. 9, 2009
(86) PCT No.: PCT/EP09/66773

§ 371 Date: Jun. 9, 2011

(30) Foreign Application Priority Data
Dec. 10, 2008 (FR) 0858457

(51) Int. Cl. A61K 49/18; A61P 43/00

(52) U.S. Cl. 424/9.361

(57) ABSTRACT

The present invention relates to the use of a CEST contrast agent in a method of CEST imaging, wherein the contrast agent is a composition comprising an encapsulating system ES encapsulating at least one CEST agent, wherein the at least one CEST agent is constituted of a monomeric chelate of a chelate of q≧2 type, or of a multimer of monomeric chelates of q≧2 type, and wherein said chelate is free inside the encapsulating system.

The application relates to contrast agents for CEST imaging.

Patent WO 2006032705 describes contrast products intended for CEST imaging, comprising encapsulating systems of lipid nanoparticle type (in particular liposomes and double emulsions) capable of including an aqueous phase and having an effect in CEST imaging. These encapsulating systems (ES) of WO 2006032705 constitute a major innovation recognized by the scientific community of contrast products through the use of a CEST mechanism associated with a flow of a high number of protons compared with the prior systems. They comprise, associated with their membrane or free inside the compartment delimited by this membrane, chelates capable of chelating shift-effect metal ions (also possibly denoted CEST-effect metals). CEST agents are also denoted for convenience in the application shift agents or shift CEST agents. Shift-effect metals (used in CEST imaging) are known to those skilled in the art; they are in particular the following elements: iron(II), Cu(II), Co(II), erbium (II), nickel (II), europium (III), dysprosium (III), gadolinium (III), praseodynium (III), neodinium (III), terbium (III), holmium (III), thulium (III), ytterbium (III).

The need remains to further improve the sensitivity of such CEST contrast agents. In particular, it is not always easy to clearly distinguish the CEST-imaging signal of such ES CEST systems, associated with a certain measured value for chemical shift Delta, compared with the endogenous signal of molecules, in particular of proteins carrying amide functions. It is recalled that this endogenous signal corresponds to a Delta shift less than or equal to a value close to 4 ppm at the appropriate irradiation frequencies (a spectrum is obtained with a peak for the endogenous water of between −4 and +4 ppm, and even higher due to T2* relaxation).

It is thus a question, for those skilled in the art, of succeeding in obtaining a distinctive signal for the contrast product injected into the patient, compared with the endogenous signal curve, and in particular of sufficiently increasing the Delta value for the product, so as to obtain a value significantly higher than approximately 4 ppm for the Delta (chemical shift difference between intra-ES and extra-ES water protons).

The publication Chem. Commun., 2008, 600-602 describes two main lines for improving liposome systems for CEST imaging (lipocest). For a good understanding of the application it is introduced here that, as well known for the one skilled in the art (see for instance Chem. Rev.1999, 99, 2293-2352, Caravan et al, notably page 26), chelates of lanthanides have a certain q value which corresponds to the number of water molecules in a situation of exchange with the chelate. For instance chelates such as DOTA or HP-DO3A have a value q equal to 1, and are designated q=1 chelates, whereas chelates such as PCTA or DO3A are a q=2 chelates.

A first main line consists in using chelates, no longer in the form of monomers, but in the form of neutral (uncharged) multimers. The principle of using multimers is advantageous since it makes it possible to increase the Delta shift without increasing the osmolality of the product. This is because compounds injected into the patient should have a sufficiently limited osmolality (number of elements inside the liposome) for the liposome to remain stable in vivo, with an osmolality inside the encapsulating system that is sufficiently close to those of biological fluids, in particular of blood (300 mOsm/Kg): if the difference between the osmolality inside and outside the system is too great, there is a risk that said system will become deformed and be damaged (explosion or implosion, in particular). Concretely, this publication shows (in particular page 601 and FIG. 1) that, at the maximum concentrations accessible inside the spherical liposome, there is thus a change of the Delta shift value from 3.5 ppm with a monomer of HP-DO3A-Tm (chelate of q=1 type) to 6.1 with a dimer formed by assembling two monomers of HP-DO3A, and approximately 9 with a trimer formed by assembling two monomers of HP-DO3A and one DTPA. Thus, in order to obtain a target signal of 20 ppm for example, it would be necessary to use a hexamer of HP-DO3A chelates, for example.

Now, the possibility of multimerization is limited in pharmaceutical industrial practice since the multimers have the drawback of being complex and expensive to produce at an industrial level, in particular above the trimer or the tetramer. This difficulty is all the more bothersome since the degree of incorporation in the liposome encapsulating system is low, only of the order of 10%, which amounts to saying that it is necessary to “discard” 90% of the multimers. Consequently the use of multimers is still advantageous, but there is a need of obtaining notably multimers that will lead to a good signal while being not to complex to make, and that provide a good efficiency, in particular a high value of shift (Delta shift in ppm) per monomer.

A second main line, where appropriate cumulative with the first main line, comprises the use of liposomes that are no longer spherical, but deformed (called shrunken), obtained by applying an osmotic shock and using appropriate membrane constituents. This solution may be attractive since the obtained shift described is multiplied by a factor of the order of 3 to 4 compared with the spherical liposomes, with a value of about 21 ppm for a dimer of HP-DO3A, for example, and about 28 ppm for a trimer comprising two HP-DO3A and one DTPA.

However, these are results on the laboratory scale, and this pathway poses several problems in clinical practice:
the complexity of production at an industrial level, and the stability of the liposome thus deformed, during months of storage in a pharmaceutical contrast product to be injected into the patient;
the risk of loss of effectiveness in vivo once the product has been injected into the patient: in a physiological medium, the deformed liposomes are exposed in vivo to osmolality/osmotic pressure conditions that may significantly or even completely alter this particular form, and to magnetic field conditions that may degrade the required “BMS” effect (bulk magnetic susceptibility effect) associated to these systems, hence a high risk of instability, and therefore of loss of effectiveness of the product for its use in practice in humans;
a reduced signal quality, and in particular a widening of the CEST spectral lines;
a loss of internal water content which diminishes the CEST signal per liposome.

Consequently, in view of the whole prior art, the invention aims to obtain encapsulating systems, in particular but not limitatively liposomes, which, on the one hand, are sufficiently stable and suitable for use in CEST imaging in the patient, and, on the other hand, when multimeric chelates are used, make it possible to increase the Delta shift (the value R) per monomer. In particular a problem to be solved is to obtain encapsulating systems ES that provide a Delta shift (R per monomer) much higher than the prior art values which are about 4 for spherical ES and about 10 for non spherical ES.

Encapsulating systems are particularly sought for CEST imaging which make it possible to obtain appropriate physicochemical values of osmolality (advantageously between 200 and 400, preferably between 250 and 350 mOsm/Kg), stability (several months), viscosity, of the products to be injected to the patient.

The applicant has now shown that by using specifically monomeric chelates q≧2 (and notably q=2) and/or mutimers of monomeric chelates q≧2, the imaging result in vivo is remarkably improved, as compared to using chelates q=1, although this was not expected for reasons detailed below.

It is, moreover, specified that document WO 2006032705 describes precisely (example 3) encapsulating systems incorporating chelates of PCTA type (which have a theoretical value for the number q of exchanged water molecules equal to 2) which are rendered lipophilic for their attachment to the liposome membrane. Approximately 100% of the PCTA chelates are thus attached to the membrane. Approximately 50% of these PCTA chelates are attached to the membrane with the hydrophilic part of the chelate, represented by the macrocyclic core, being directed toward the inside of the system; and 50% of these PCTA chelates are attached to the membrane with the hydrophilic part of the chelate, represented by the macrocyclic core, being directed toward the outside of the encapsulating system. These exemplified chelates are thus not able to move freely within the encapsulating system, such free movement requiring chelates not bearing lipophilic groups for attachment to the membrane of the encapsulating system. Advantageous effect of free chelates is explained below. It is, moreover, specified that the document Chem. Commun., 2008, 600-602 and the other known associated publications of the applicant describe only multimers of monomeric chelates, the monomers of which are of q=1 type.

To this effect, according to a first aspect, the invention relates to a composition for CEST imaging, comprising an encapsulating system ES encapsulating at least one CEST agent (also denoted shift agent) constituted of a monomeric chelate of a chelate of q≧2 type, or of a multimer of monomeric chelates of q≧2 type, and said chelate being free inside the encapsulating system.

The invention in particular relates to the use of a contrast agent in a method of CEST imaging, the contrast agent being a composition comprising an encapsulating system ES encapsulating at least one shift agent, wherein the at least one shift agent is constituted of a monomeric chelate q≧2, or of a multimer of monomeric chelates q≧2, and wherein said chelate is free inside the encapsulating system.

It is precised that the expression “shift agent is constituted of a monomeric chelate q≧2” means that the shift agent is advantageously the complex of a shift-effect metal ion with the q≧2 monomeric chelate, i.e. the monomeric complex of the metal with the monomeric chelate.

It is precised that the expression “shift agent is constituted of a multimer of monomeric chelates” means that the shift agent is advantageously the complex of a shift-effect metal ion with the multimer of monomeric q≧2 chelates.

It is also reminded that the ES systems liposomes and double emulsions encapsulate a pool of water mobile protons to be shifted.

The invention also relates to a method of Cest imaging using a composition comprising an encapsulating system ES encapsulating at least one shift (CEST) agent wherein the at least one shift (CEST) agent is constituted of a monomeric chelate q≧2, or of a multimer of monomeric chelates q≧2, and wherein said chelate is free inside the encapsulating system.

The applicant has obtained nanosystems for CEST in which the value of the ratio R (signal efficiency) between the Delta shift (ppm) and the number of monomeric chelate (and thus by lanthanide) is about 8.

In the prior art of spherical ES for Cest imaging:
for a monomeric chelate q=1, the ratio R is about 4/1=4
for a dimer made of two monomeric chelates q=1, the ratio R is 8/2=4

Whereas thanks to the use of monomers of chelates q=2:
for a monomeric chelate q=2, the ratio R is about 8/1=8
for a dimer made of two monomeric chelates q=2, the ratio R is 16/2=8.

In practical terms, for spherical ES, in order to obtain a Delta shift of 8 ppm, the applicant can use a monomer instead of a dimer; and to obtain a Delta shift of more than 20 ppm, the applicant can use a trimer instead of a hexamer.

Advantageously the invention also relates to a composition for CEST imaging (comprising an encapsulating system ES encapsulating at least one shift CEST agent constituted of a monomeric chelate of a chelate of q≧2 type, or of a multimer of monomeric chelates of q≧2 type, and said chelate being free inside the encapsulating system), and for which ratio R of Delta shift (δ ppm) by the number n of monomeric chelates (and thus by lanthanide) is more than 7 preferably of about 8 or more than 8.

R=(δ/nₘₒₙₒₘₑᵣₛ)≧7

In particular the applicant has succeeded in obtaining spherical systems, in particular liposomes, which are thus at least twice as effective as the prior spherical systems such as those described in Chem. Commun., 2008, 600-602.

Advantageously, the shift CEST agent is completely encapsulated in the aqueous phase of the encapsulating system. The expression “chelate free inside the encapsulating system” is intended to mean that the chelate does not bear any anchoring groups (lipophilic groups, in particular) which enable covalent bonding with the inner membrane of the encapsulating system. The chelate is not therefore covalently bonded to the encapsulating system, in particular to the wall of the encapsulating system. In particular, this chelate is not modified by the addition of a lipophilic group as described in example 3 of patent application WO 2006032705 or of the phospholipid type or comprising a long carbon chain (for example, more than 15 to 20 carbon atoms). Advantageously, it is therefore a hydrophilic chelate, i.e. a chelate that is soluble in an aqueous phase.

In the application, the term “monomeric chelate of q≧2 type” or more simply “monomeric q≧2 chelate” denotes any monomeric chelate having an exchange of q water molecules in the internal sphere or the second sphere of the monomeric chelate, with q≧2. This includes the case of chelates of q=n type, n being greater than 2, notably q=3. Thus for instance:
a monomeric chelate q=2 is a chelate (monomer) having q=2
a dimer of a monomeric chelate q=2 is a chelate made of two monomeric chelates each having q=2, and linked together by a chemical link
a dimer of a monomeric chelate q=3 is a chelate made of two monomeric chelates each having q=3, and linked together by a chemical link.

The applicant has made an invention of selection with a motivated choice by using among the huge quantity of chelates of the prior art a specific category of chelates that are monomeric chelates q≧2, or multimer of monomeric chelates q≧2, and that are free inside the ES (the q=2 chelates disclosed in WO2006032705 is a lipophilic chelate attached to the membrane and therefore is not free inside the ES; the other chelates described as free chelates encapsulated in the ES of the prior are presented as a general global category; the examples detailed in the prior art are q=1 chelates and multimers of q=1 chelates).

It is now emphasized that and explained why for the one skilled in the art of chelates for CEST agents, the such high efficiency of monomeric chelates q=2 in particular for nanosystems was not obvious. In summary as described in detail hereafter, the one skilled in the art would have expected that a q=2 monomeric chelate would not be more efficient (R ratio) than a q=1 monomeric chelate. The one skilled in the art would have tested different associations/combinations of chelates q=1 instead of focusing on monomeric chelates q≧2 or on multimers of monomeric chelates q≧2.

From a basic point of view, one may consider that some multimeric chelates are equivalent to a sum of monomers, for instance that a dimeric chelates of two chelates q=1 should be equivalent to a chelate q=2.

But the one skilled in the art knows that the physics of shift agents is much more complex than this hypothesis. More precisely, it was known notably from Chem. Soc.Reviews, 1998, vol 27, pages 19-28 (in particular page 26) and from Progress in Nuclear Magnetic Resonance Spectroscopy, 28, (1996), 283-350, that monomers of chelates need to have a proper conformation so that a favourable shift (CEST) effect in obtained.

Indeed physical models and molecular projections in the prior art show that an efficient shift effect requires a certain position of the water molecules towards the chelate and the lanthanide. More precisely, as shown in FIG. 5c and FIG. 6, the lanthanide and the (one) water molecule are in favourable conditions when the water is positioned in the cone (in the area delimited by the solid angle ψ) relative to the plan, as determined by a term of the type (3 cos²θ-1)/r3, where r and θ are the polar coordinates of the nucleus with respect to the lanthanide ion and with the main magnetic axis.

By extension dimers (having q=2 for the whole chelate) of chelates q=1 that are represented in FIG. 7, and more generally multimers of chelates q=1, would be appropriate considering that the monomers are linked together in a conformational way so that the water molecule of each monomer has a proper position in the space. The structure shown (the solid angle) is substantially obtained for each monomer q=1. Thus the “efficient position” of the lanthanide and the water molecule are presumably obtained for each monomer, leading to an additive efficiency for the whole multimer.

In fact, the situation is different between monomeric chelates having q=1, and monomeric chelates having q>1.

For monomeric chelates q=2 notably, the monomeric chelate q=2 and the lanthanide are in water molecule exchange for two water molecules. As shown in FIG. 8, the desired conformation was expected not be met for each water molecule since the second water molecule is far from the desired position (close to the magnetic symmetry axis and inside the cone): more precisely, one water molecule is axial, whereas the other water molecule is equatorial (see the projections for DO3A chelate q=2 represented in FIG. 5a and FIG. 5b extracted from Chem.Eur.J, 2003, 9, 5468-5480). This second equatorial H2O molecule is thus not at the presumed good position. And thus according to the prior art the q=2 monomer should not have been better than a q=1 monomer.

The FIG. 5b presents the angular projections of the inner-sphere water oxygen atom and its neighbouring coordination sites for a monomeric chelate DOTA (q=1) and a monomeric chelate DO3A (q=2). The shape described by the atoms around the water molecules for [Gd(DOTA)(H₂O)] and [Gd(DO3A)(H2O)2](a) centered on axial axis OWA is close to a square. The shape around [Gd(DO3A)(H2O)2](b) centered on OWB is close to a pentagon, and the solid angle w is significantly larger than for [Gd(DO3A)(H2O)2](a).

Thus, surprisingly, the results obtained with the compounds of the applicant, as illustrated in FIG. 9, imply that the two water molecules in exchange with the monomeric chelates q=2 act as if they were located in the optimal cone.

It is also emphasized in a complementary approach that, for a high efficient use in nanosystems and in particular liposomes, the chelates q=2 of the applicant need that the two water molecules, first have a very fast exchange water, and second have substantially the same behaviour (to avoid a kind of mismatch/misbalance between the two water molecules). This was not expected notably from Chem.Eur.J, 2003, 9, 5468-5480 that indicates “If the area around the axial water is smaller than the area around the equatorial water, the water exchange rate of the axial water should be higher than the equatorial one”.

Further the molecular behaviour of water molecules is different between monomeric chelates q=1 and monomeric chelates q=2 since chelates q=2 have a water cinetic of the associative type whereas chelates q=1 have a water cinetic of the dissociative type, which could have lead to such misbalance.

Consequently in view of the prior art of shift (CEST) imaging, a monomeric chelate q=2 was not expected to be particularly more satisfying than a monomeric chelate q=1. In particular for chelates q=2 it was not at all obvious that a monomeric chelate q=2 free in the encapsulating system would be so efficient (R ratio in particular) and notably similar or better to a dimer made of two monomers of chelates q=1.

It is further explained that the q=2 free monomeric chelates of the ES (and their multimers) of the applicant do no behave like the q=2 lipophilic (attached to the membrane of the ES/liposome) described in WO2006032705 (example 3, PCTA compound). The technical effect on shift is very different between chelates that are free in the ES and membranar lipophilic chelates. More precisely, for a given concentration of ES administered to the patient (liposome formulation for instance), the concentration of chelates available for shift will be much higher for free q≧2 chelate than for lipophilic membrane integrated q≧2 chelates. The concentration of free q≧2 monomeric chelate is about 300 mM for example as explained later in the application, whereas the concentration of lipophilic q≧2 chelates is about only 1 to 10 mM and typically 2 to 5 mM (the lipophilic chelate is only a part of the components of the membrane typically about 10%) leading thus to a low shift. Consequently the Delta shift of the ES (in particular liposomes) encapsulating water is much better with the free q≧2 monomeric chelates or free multimers of monomeric q≧2 chelates. Advantageously, the monomeric q=2 chelate used is chosen from the monomeric chelates known and having q=2: PCTA, DO3A, DO3MA, AAZTA and HOPO (the hydroxypyridinone HOPO that are q=2), and derivatives thereof; preferably PCTA or DO3A.

Advantageously, the monomeric q=3 chelates are chosen from the monomeric chelates known and having q=3: HOPO (more precisely the hydroxypyridinone HOPO that are q=3), PC2A, BP2A, TX (texaphyrin), NOVAN and N6-L1, and derivatives thereof, advantageously from HOPO, PC2A, BP2A and Tx. Among the q≧2 derivatives known from the prior art, mention will in particular be made of the compounds in the following table, those described in Chemical Reviews, 1999, vol 99, No. 9, 2293, and any derivative that can be predicted by chemoinformatics, the method of prediction being described, for example, in Inorg Chem, 1996, 35, 7013-7020, Bioconjugate Chem, 1999, 10, 958-964, and Bioconjugate Chem, 2004, 15, 1496-1502.

| MONOMERIC | | |
|---|---|---|
| CHELATE | Value q | Reference |
| PC2A | 3 | Chem Reviews, 1999, 99, 9, p 2328-2329 |
| PCTA and | 2 | Chem Reviews, 1999, 99, 9, p 2328-2329 |
| derivatives | | and Coordination chemistry Reviews, 251, |
| | | 2007, 2428-2451 compounds 47, 48, 50 |
| BP2A | 3.5 | Chem Reviews, 1999, 99, 9, p 2328-2329 |
| NOVAN | 4 | Chem Reviews, 1999, 99, 9, p 2328-2329 |
| Tx | 3.5 | Chem Reviews, 1999, 99, 9, p 2328-2329 |
| N6-L1 | 3 | Chem Reviews, 1999, 99, 9, p 2328-2329 |
| DO3A and | 2 | Inorg. Chem, 1996, 35, p7013-7020 and |
| derivatives | | Coordination chemistry Reviews, 251, 2007, |
| | | 2428-2451, compounds nos |
| | | 13, 14, 21, 27, 28, 30, 31, 32, 33, 34, 35-36, |
| | | and compounds 53, 54, 55, 56 |
| DO3MA | 2 | Inorg. Chem, 1996, 35, p7013 |
| MeDTPA | 2 | Inorg. Chem, 1996, 35, p7013 |
| MeDTA | 2 | Inorg. Chem, 1996, 35, p7013 |
| Me2DETA | 2 | Inorg. Chem, 1996, 35, p7013 |
| NOTMA | 2 | Inorg. Chem, 1996, 35, p7013 |
| DO2A | 3 | The Chemistry of contrast agents in medical |
| | | MRI, A. Merbach, 2001, chap 2, p44-119, |
| | | relaxivity of gadolinium III complexes |
| PDTA | 2 | The Chemistry of contrast agents in medical |
| | | MRI, A. Merbach, 2001, chap 2, p44-119, |
| | | relaxivity of gadolinium III complexes |
| TTAHA | 2 | The Chemistry of contrast agents in medical |
| | | MRI, A. Merbach, 2001, chap 2, p44 |
| Taci | 2 | The Chemistry of contrast agents in medical |
| | | MRI, A. Merbach, 2001, chap 2, p44 |
| Bipyridine | 3 | Coordination chemistry Reviews, 251, 2007, |
| compound 51, 52 | | 2428-2451 |
| and derivatives | | |
| Compounds of | 2 to 3 | Angewandte Chem Int Ed, 2008, 47, 2 to 15 |
| HOPO type and | | Chelates of the compounds of FIG. 6 |
| derivatives | | |
| AAZTA | 2 | Bioorganic & Medicinal Chemistry Letters, |
| | | 19, 2009, 3442 |
| | | Organic & Biomolecular Chemistry, 2009, |
| | | 7, 1120-1131 |
| | | Inorganica Chimica Acta, 361, 2008, 1534- |
| | | 1541 |

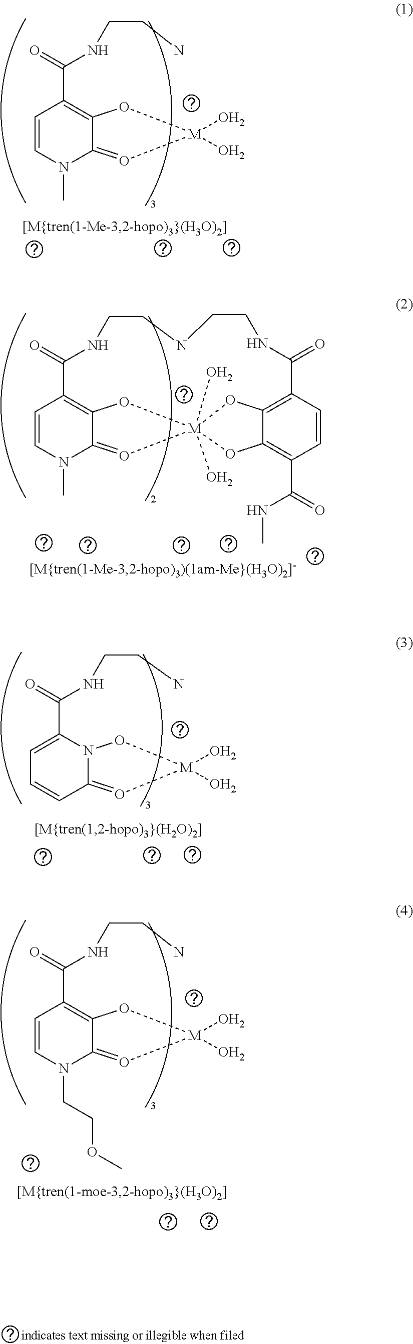

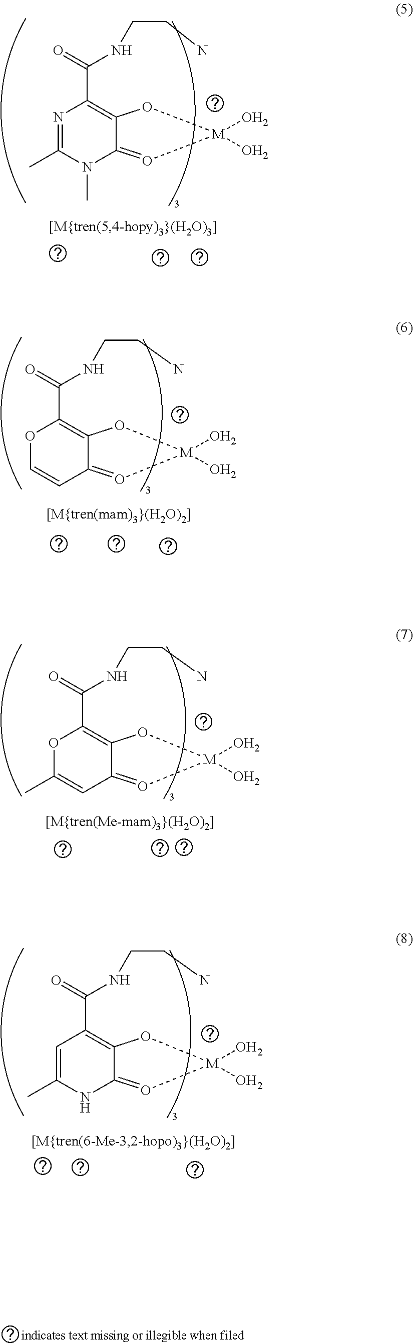

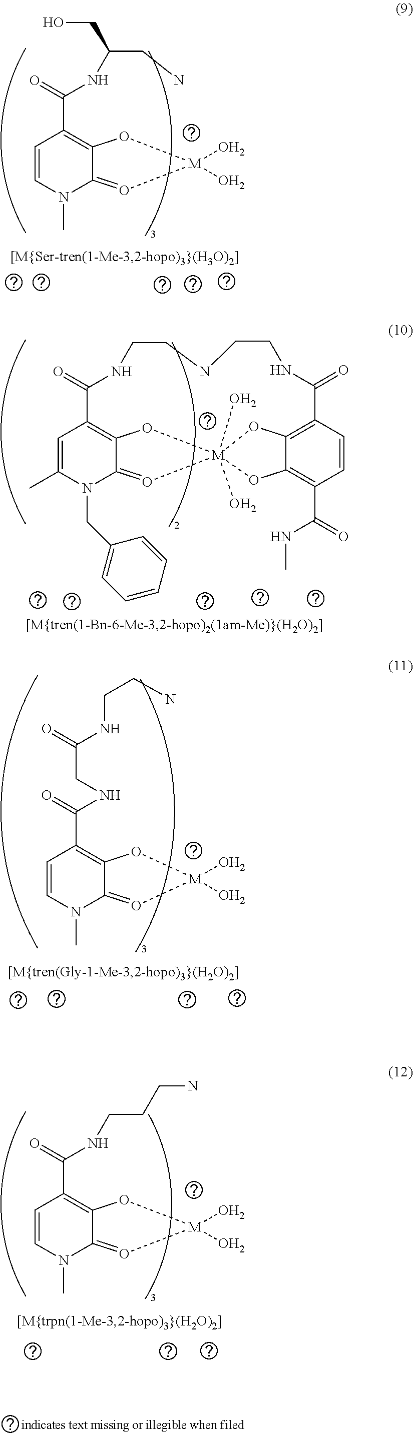

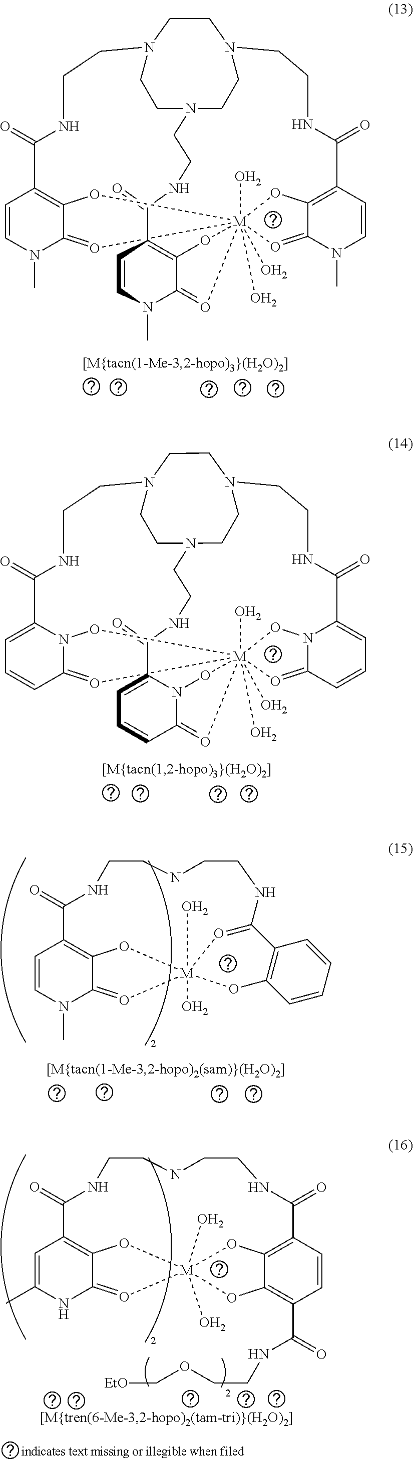

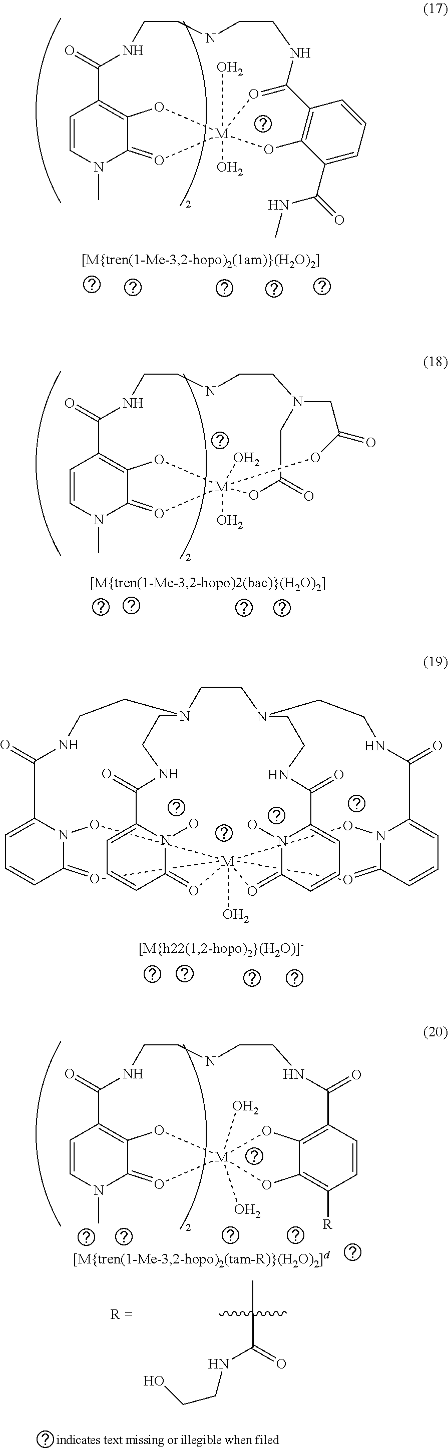

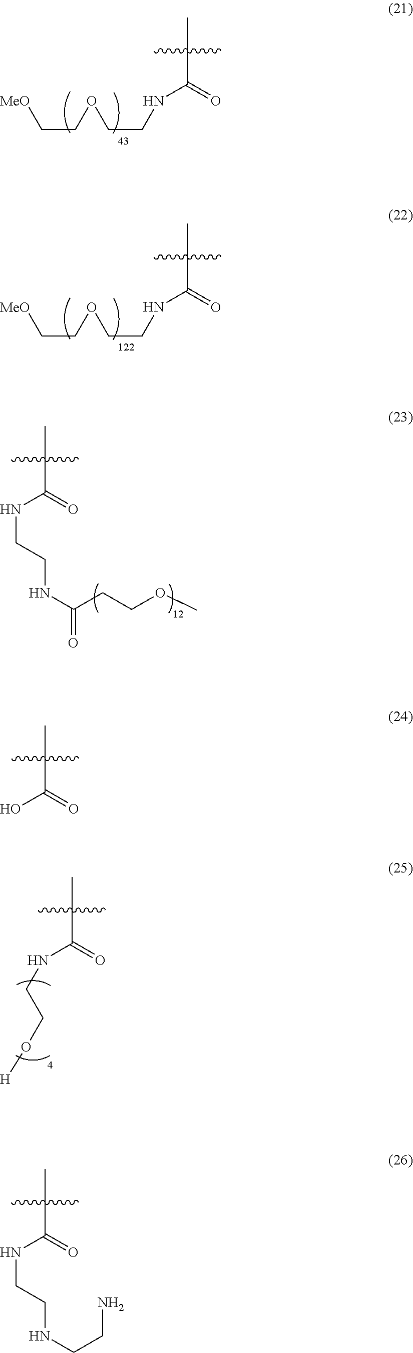

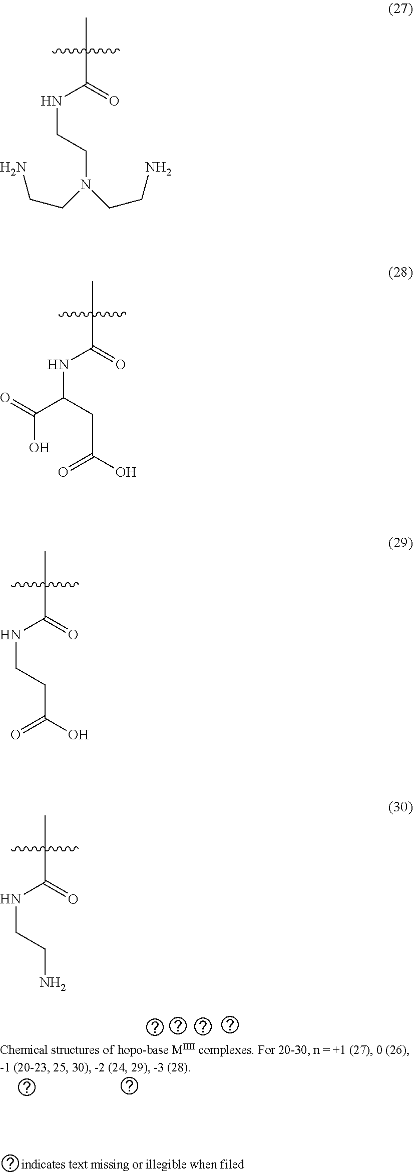

Advantageously, the shift-effect metals of the shift agent are chosen from the following: iron(II), Cu(II), Co(II), erbium (II), nickel (II), europium (III), dysprosium (III), gadolinium (III), praseodynium (III), neodinium (III), terbium (III), holmium (III), thulium (III) and ytterbium (III); and preferably from: Dy3+, Tb3+, Tm3+, Yb3+, Eu3+, Gd3+, Er3+ and Ho3+, in particular from: Dy3+, Tb3+, Tm3+,Yb3+, Eu3+ and Gd3+.

Advantageously, the chelates are chosen from PCTA-Tm, DO3A-Tm, HOPO-Tm, AAZTA-Tm, PCTA-Dy, DO3A-Dy, HOPO-Dy and AAZTA-Dy, advantageously from PCTA-Tm, PCTA-Dy, DO3A-Tm and DO3A-Dy, DO3A-Yb.

In the application, the term “encapsulating system” (ES) denotes liposomes or any other system capable of including an aqueous phase containing a CEST agent (chelate associated with a metal that is active in CEST) which makes it possible to provide an effect in CEST imaging, which includes in particular liposomes (lipid biolayer), water/oil/water double emulsions, water-in-oil emulsions, in an organic solvent (lipid monolayer with a polar portion facing the inside of the micelle so as to make an aqueous space in the compartment delimited by the micelle). The definitions of these systems are well known and are, for example, summarized at http://en.wikipedia.org/wiki/Micelle.

Thus, advantageously, the term “liposome” is intended to mean a vesicle, the center of which is occupied by an aqueous cavity and the shell of which is constituted of a varying number of phospholipid-based, bimolecular sheets. Advantageously, the liposomes are spherical liposomes, but non spherical deformed vesicles mays also be used.

The liposomes may be multilamellar, i.e. may comprise several concentric compartments and several walls (lamellae or sheets). Advantageously, the diameter of the aqueous cavity ranges from 100 to 300 nm, the distance between the sheets is of the order of 1.8 nm and the thickness of each sheet ranges from 4.8 to 6.9 nm, with a total diameter of between 0.4 and 3.5 micrometers. Advantageously, the liposomes according to the present invention have a diameter, in the case of the spherical liposomes, or a largest dimension, in the case of the nonspherical liposomes, of between 20 and 500 nm, advantageously between 20 and 200 nm.

Advantageously, the liposomes are obtained from phospholipids. They are in particular described in patent application WO 2006032705. They can form spontaneously by dispersion of lipids, in particular of phospholipids, in an aqueous medium by conventional techniques such as those described in WO92/21017, which includes sonication, homogenization, microemulsification and spontaneous formation by hydration of a dry lipid film.

Other advantageous ES are double emulsions. Double emulsions refer to emulsions water/oil/water being dispersions of oily globules in which water drops have been prior dispersed. Advantageously, two surfactants used for the double emulsion are such that their respective HLB allows the formation of the globules, one surfactant with high HLB, the other surfactant with low HLB. The ratio is such that the efflux of water can be controlled between the inside and the outside of the globules. Double emulsions are for instance described in “how does release occur?” Pays K, Giermanska-Kahn J, Pouligny B, Bibette J, Leal-Calderon F, J Control Release. 2002 Feb. 19; 79(1-3):193-205, and in “Double emulsions: a tool for probing thin-film metastability” Pays K, Giermanska-Kahn J, Pouligny B, Bibette J, Leal-Calderon F, Phys Rev Lett. 2001 Oct. 22; 87(17):178304.

In particular, for the purpose of the present invention, the term “micelle” is intended to mean a spheroidal aggregate of molecules having a hydrophilic polar head directed toward the aqueous solvent and a hydrophobic chain directed toward the inside. The inverse micelles have, for their part, a hydrophobic chain directed toward the organic solvent and a hydrophilic polar head directed toward the inside.

Other advantageous ES systems are polymersomes, for instance described in WO2009072079 (described in detail notably pages 6, 19, 20). The term “polymersomes” is used here to generally indicate nanovesicles or microvesicles comprising a polymeric shell that encloses a cavity. These vesicles are preferably composed of block copolymer amphiphiles. These synthetic amphiphiles have an amphiphilicity similar to that of lipids. By virtue of their amphiphilic nature (having a more hydrophilic head and a more hydrophobic tail), the block copolymers will self-assemble into a head-to-tail and tail-to-head bilayer structure similar to liposomes. The amphiphilic nature of the block copolymers is preferably realized in the form of a block copolymer comprising a block made up of more hydrophilic monomeric units (A) and a block made up of more hydrophobic units (B), the block copolymer having the general structure AₙBₘ, with n and m being integers of from 5 to 5000, preferably 10 to 1000, more preferably 10 to 500. Any of the blocks can itself be a copolymer, i.e. comprise different monomeric units of the required hydrophilic respectively hydrophobic nature. It is preferred that the blocks themselves are homopolymeric. Any of the blocks, in particular the more hydrophilic block, may bear charges. The number and type of charges may depend on the pH of the environment. Any combination of positive and/or negative charges on any of the blocks is feasible. In view of the applicability in agents for medical diagnostics and treatment, it is preferred that the polymeric blocks are made of pharmaceutically acceptable polymers. Examples hereof are e.g. polymersomes as disclosed in US 2005/0048110 and polymersomes comprising thermo-responsive block co-polymers as disclosed in WO 2007/075502. Further references to materials for polymersomes include WO 2007081991, WO 2006080849, US 20050003016, US 20050019265, and U.S. Pat. No. 6,835,394.

Other advantageous ES systems are capsules such as red blood cells and derivatives thereof. The prior art discloses describes in detail the way to prepare these systems and thus the disclosure is sufficient for the one skilled in the art to prepare the systems encapsulating the chelates. Erythrocytes structures used for CEST are described in WO2009060403 pages 13-14 (detailed examples 1 and 2). Intrinsically non-spherical carriers can be based on erythrocytes, by employing erythrocyte ghosts. In order to provide a semipermeable shell that encloses a cavity comprising an MR analyte, erythrocytes are used that have lost most, and preferably all, of their original water-soluble contents. The resulting, MR analyte-containing erythrocytes are more appropriately referred to as erythrocyte ghosts. Thus, particles result in which an MR analyte is contained in a membrane which happens to be the phospholipid bilayer originating from an erythrocyte. The MR analyte-loaded erythrocyte ghosts are obtainable by a process comprising the steps of providing erythrocytes, subjecting the erythrocyte to hypotonic lysis so as to provide an opening in the erythrocyte membrane, subjecting the opened erythrocyte to one or more washing steps so as to substitute a medium being the MR analyte (such as water), or a solution or dispersion of an MR analyte (such as metabolites dispersed or dissolved in water), or any other liquid comprising a desired MR analyte, for at least part of the original water-soluble remove contents of the erythrocyte, and subjecting the resulting MR analyte-loaded erythrocyte ghosts to a closing step under isotonic conditions.

Depending on embodiments, the encapsulating system (advantageously liposome system) comprises several identical or different CEST agents. Thus, this encapsulating system encapsulates at least one monomeric q≧2 chelate, but also at least one other different chelate, for example chosen from the following categories:
a) a monomeric chelate of q≧2 type, for example another q=2 chelate;
b) a q=1 chelate.

It is possible for this other chelate not to be free inside the encapsulating system and, for example, to be associated with the membrane of the encapsulating system. Advantageously, it is free inside the encapsulating system.

Thus, the encapsulating system according to the invention thus encapsulates:
a) several chelates which are identical to or different than one another and of q≧2 type, for example, several monomeric q=2 chelates;
b) at least one monomeric q≧2 chelate and at least one q=1 chelate;
c) at least one monomeric q=2 chelate and at least one q≧2 chelate.

For each of these categories, the shift agents (i.e. the metal) are identical or different between the chelates.

Depending on embodiments, the composition comprises several encapsulating systems (advantageously liposome systems) with several different CEST agents, for example the composition is a mixture of liposomes encapsulating a chelate of q≧2 type and of liposomes encapsulating a chelate of q=2 type or q=1 type.

There will be, for example:

a) liposomes that are different in a contrast agent, for example liposomes encapsulating a first chelate with a first metal, and liposomes encapsulating another chelate with another metal; there will, for example, be a composition comprising liposomes encapsulating one or more PCTA-Tm chelates and liposomes encapsulating one or more DO3A-Ym chelates;
b) liposomes which each include different metals; there will, for example, be a composition of identical liposomes, each liposome encapsulating, for example, one or more PCTA-Tm chelates and one or more DO3A-Ym chelates or one or more PCTA-Tm chelates and one or more PCTA-Ym chelates.

It is thus understood that the various combinations are possible.

In one particular embodiment, the chelate used accordingly to the invention is a multimer (advantageously a dimer, a trimer or a tetramer) made of several monomeric chelates having q≧2, advantageously a dimer, a trimer or a tetramer of a chelate q=2.

Depending on embodiments, the chelates of q≧2 type are in the form of multimers of monomeric q≧2 chelates, for example 2, 3, 4 PCTA chelates linked to one another.

Depending on embodiments, the multimers form linear assemblies of chelates.

The linear multimers are advantageously of formula:

(Ch i)-(linker i)-(Ch j)-(linker j)- . . . -(Ch k) (I)
the chelates Ch i,j,k being monomeric q≧2 chelates which are identical to or different than one another,
the linkers i, j, k being a chemical bond or a chemical bonding group, and being identical to or different than one another.
The structure of the chelates and of the linkers is chosen in such a way that every monomeric chelate of the multimers of formula (I) have a behavior of q=2 type, the chelate Ch i,j,k being, where appropriate, functionalized for possible grafting with the linker. Linkers and functionalizations bearing atoms coordinating the first sphere of coordination of the complex will be avoided so as to avoid them leading to the chelates having a q=1 behavior.

The following will in particular be cited as linkers:
1) (CH₂)₂-phenyl-NH, (CH₂)₃—NH, NH—(CH₂)₂—NH, NH—(CH₂)₃—NH, nothing or a single bond;
2) saturated or unsaturated, linear or branched C₁-C₂₀, in particular C₃-C₁₀, alkylene, propyl, alkoxyalkylene, polyalkoxyalkylene, polyethylene glycol, cycloalkyl, alkylene interrupted with phenylene, arylene or substituted arylene, alkylidene, alcilidene, NH—C═O, —NH—CH═NH, NH—C═S, COO, OCO, O, S, squarate derivative,
   on the condition that the chelate conserves a q=2 behavior.

Use will also, for example, be made of linkers described (and the use of which for associating several chelates is described) in U.S. Pat. No. 5,446,145, columns 6-8, of the type of a hydrocarbon-based group comprising one or more polyalkylamine groups such as —NH(CH₂CH₂NH—)ⱼ, j preferably being 1 to 8), or aminopolyether groups or aminopolyalcohol groups with preferably 4 to 20 carbon atoms, or amino carbohydrate groups.

Mention is, for example, made of the linkers:
1,2-diaminoethane, 1,3-diaminopropane, 1,4-diaminobutane, 1,5-diamino-3-(2-aminoethyl)pentane, N,N′-dimethyl-1,2-diaminoethane, N,N′-dimethyl-1,3-diaminopropane, 2-hydroxy-1,3-diaminopropane, 2-amino-1,3-diaminopropane, 2,3-diamino-1,4-butanediol, 1,4-diamino-2,3-butanediol, 1,4-diaminocyclohexane, 1,4-phenylenediamine, and especially 1,1,1-tris(aminomethyl)ethane, 2,2′,2″-triaminotriethylamine, tris(aminomethyl)methane, diethylenetriamine, triethylenetetraamine, 1,3,5-triaminocyclohexane, and 1,3,5-phenylenetriamine;
2,2-dimethyl-1,3-propanediol, tris(2-hydroxyethyl)amine, 1,1,1-tris(hydroxymethyl)ethane and tris(hydroxymethyl)aminomethane.

The dimers of monomeric q=2 chelates below are advantageously provided (any lanthanide for cest imaging can be used):

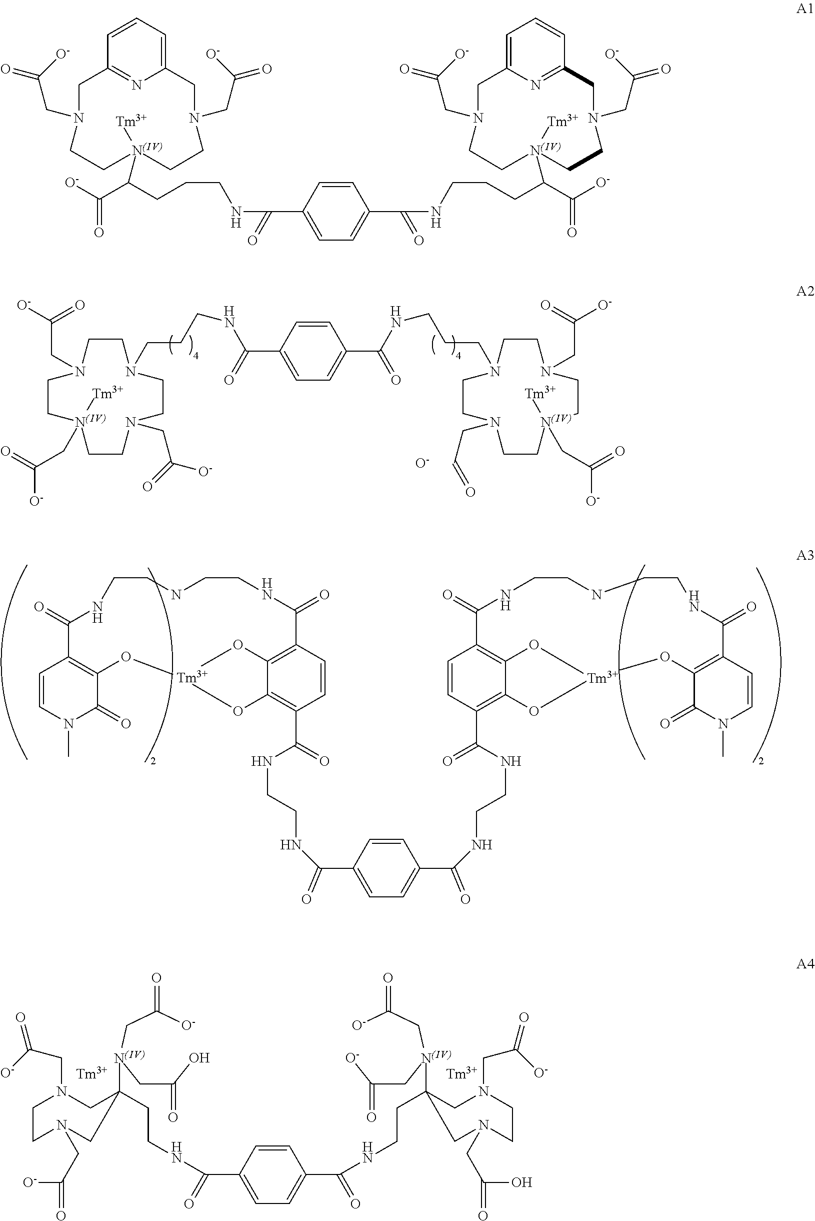

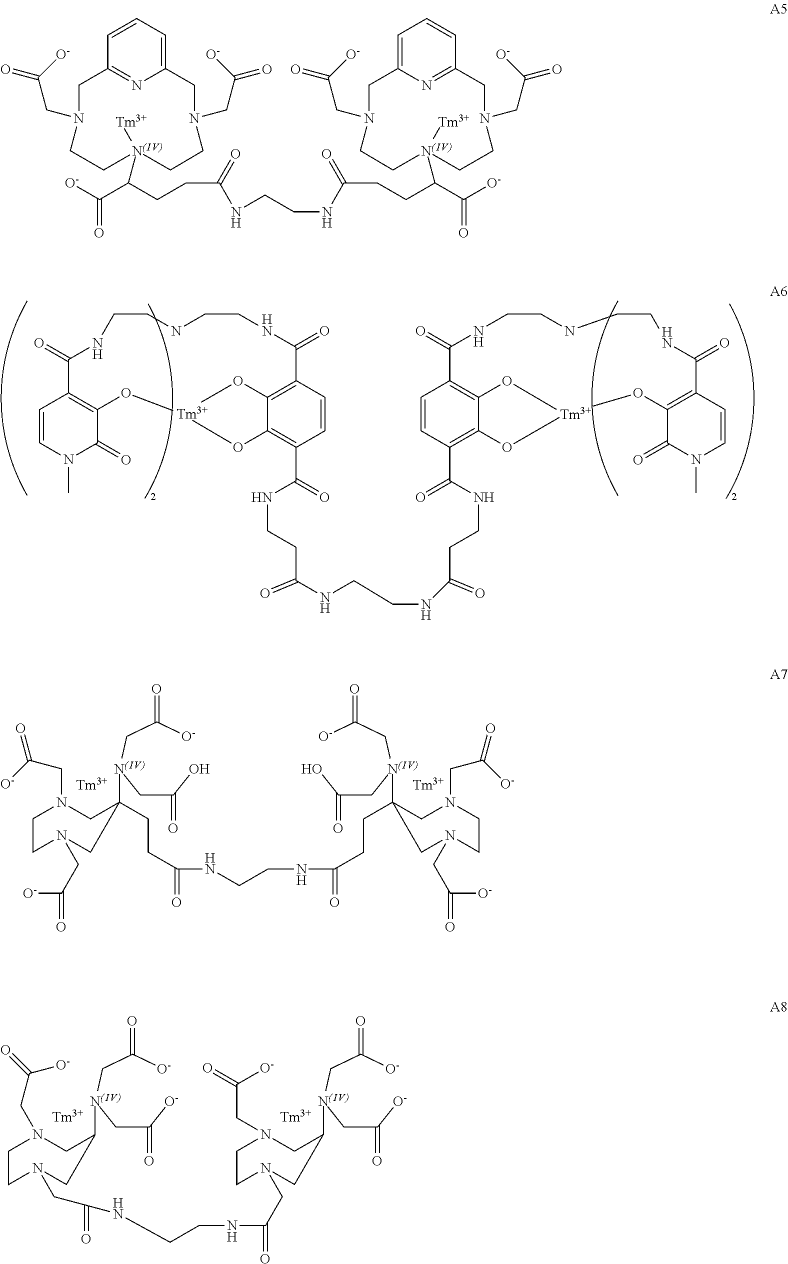

The linear trimers obtained having the formula Ch1-linker 1-Ch2-linker 2-Ch3, where Ch 1=Ch 2=Ch 3=PCTA with a metal that can be used in CEST, and linker 1=linker 2=linear or branched (C₁-C₂₀), in particular C₃-C₁₀, alkyl chain, hydroxyalkyl or arylalkyl, are also, for example, provided.

Depending on embodiments, the multimers form branched assemblies comprising several chelates grafted onto a central chemical nucleus, and in particular the compounds of formula (II):

Nucleus-(Chelate i,j,k)n (II)
with:
   Nucleus being a chemical nucleus onto which several identical or different chelates are grafted,
   Chelate being a monomeric q=2 chelate,
   n being between 2 and 6, advantageously n=2, 3 or 4,
   i, j, k being identical or different.

According to advantageous embodiments, the nucleus is:
a) for forming dimers: a diamine, for example

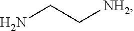

a diacid, for example

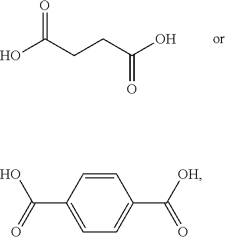

or a dibrominated compound, for example

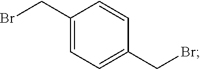

b) for forming trimers: a triamine, for example

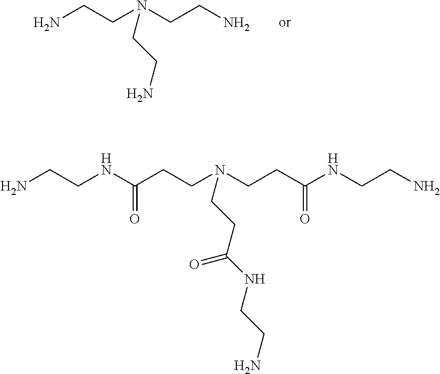

or lys-lys-lys, a triacid, for example

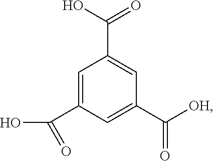

a tribrominated compound, for example

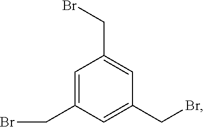

a triazine, for example 2,4,6-trichloro[1,3,5]triazine

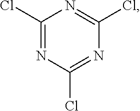

or any aromatic nucleus comprising three functions suitable for coupling with three chelates;
c) for forming tetramers: a tetramine, for example

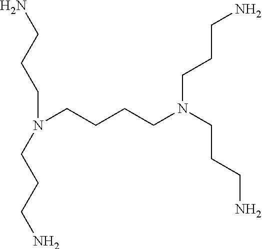

or PANAM G0

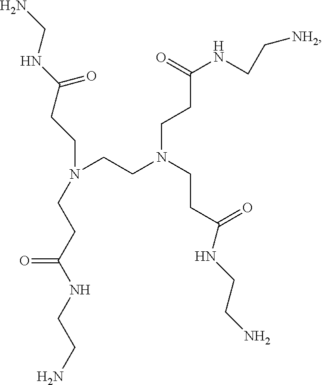

a tetrakis, for example

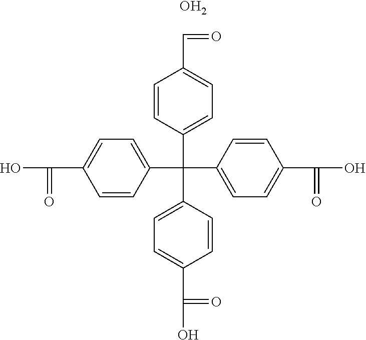

or PANAM G0.5

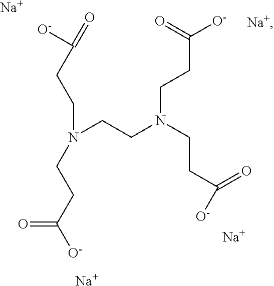

a tetrabrominated compound, for example

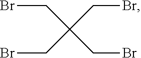

or other structures such as

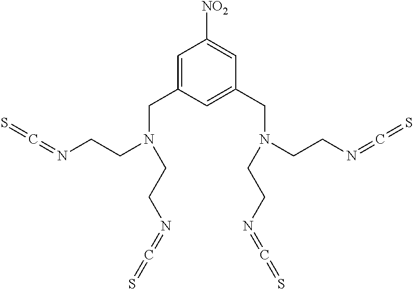

d) for forming hexamers: a phosphazine, for example

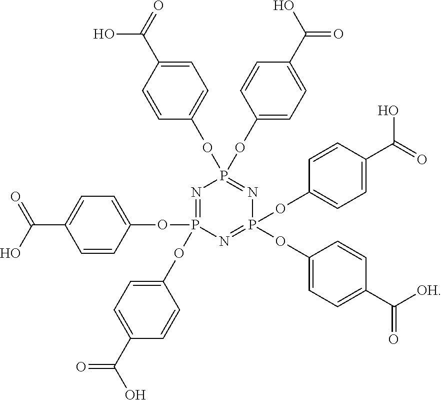

It is understood that the multimer may also have the formula Nucleus-[(linker-Chelate)] n, with n, Nucleus, linker and chelate as defined above.

As linker as above, use is made of a group chosen from:
saturated or unsaturated, linear or branched C₁-C₂₀, in particular C₃-C₁₀, alkylene, alkoxyalkylene, polyalkoxyalkylene, polyethylene glycol, cycloalkyl,
alkylene interrupted with phenylene, arylene or substituted arylene, alkylidene, alcilidene, NH—C═O, NH—CH═NH, NH—C═S, COO, OCO, O, S, squarate derivative,
polyalkylamine (such as —NH(CH₂CH₂NH—)ⱼ, j preferably being 1 to 8), or aminopolyether or aminopolyalcohol, with preferably 4 to 20 carbon atoms, or aminocarbohydrate.

The structure for the monomeric chelates q=2 and for the linkers is chosen such that the monomeric chelates have a behavior of q=2 type; the following multimeric compounds of q=2 chelates are thus obtained:

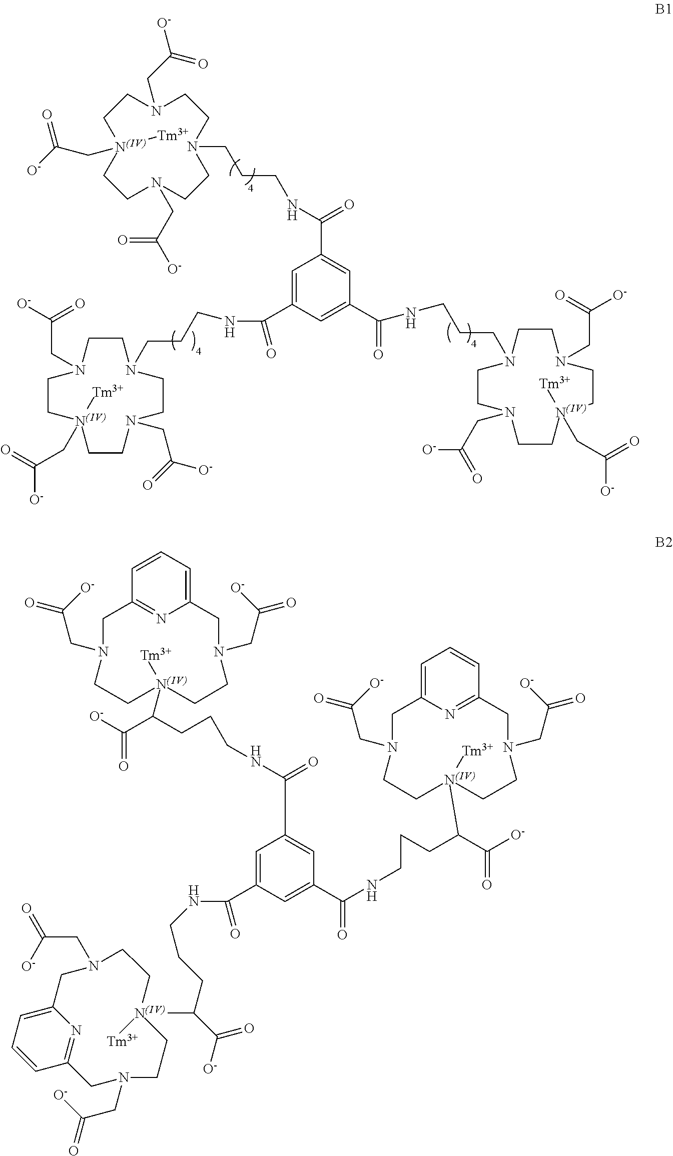

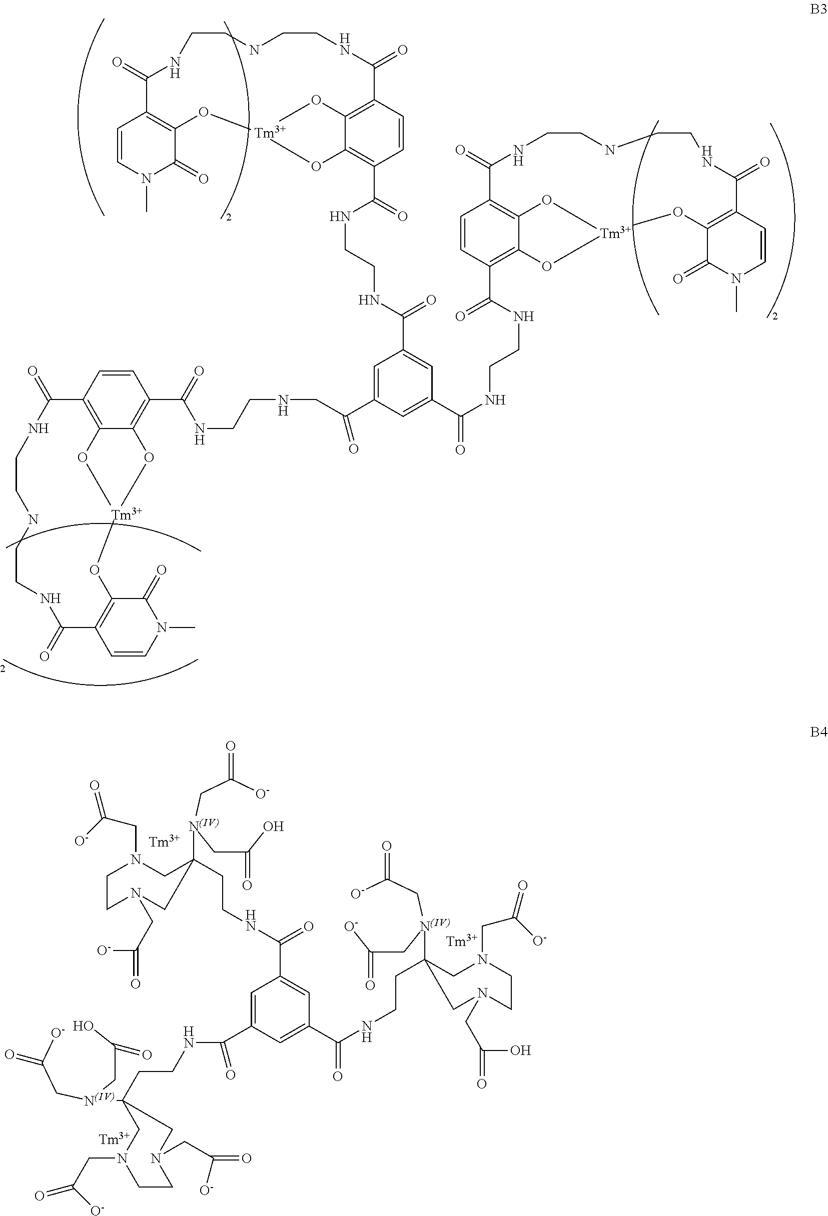

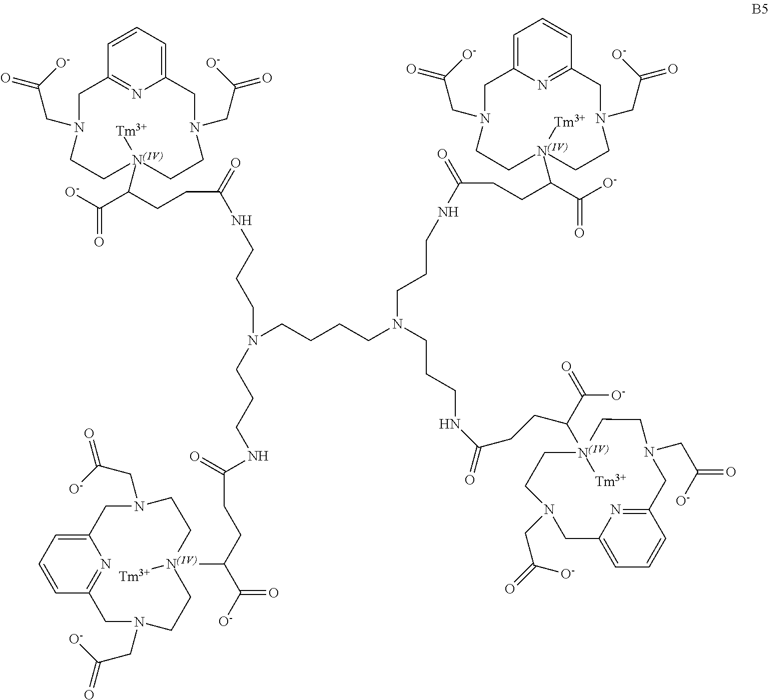

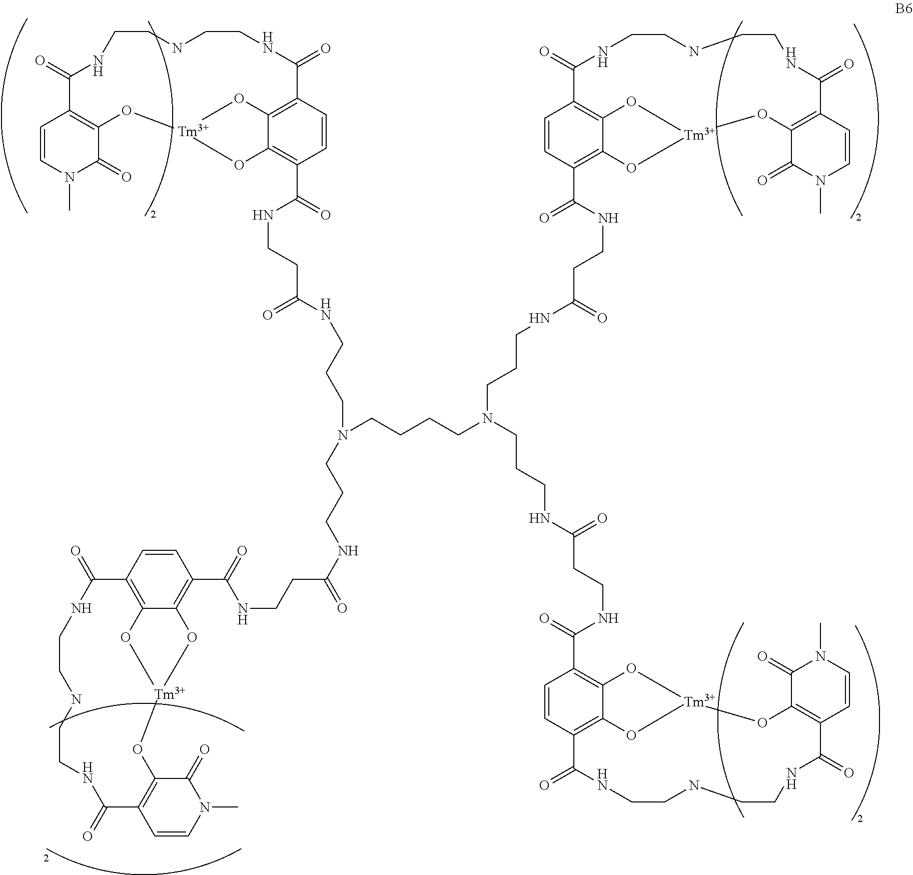

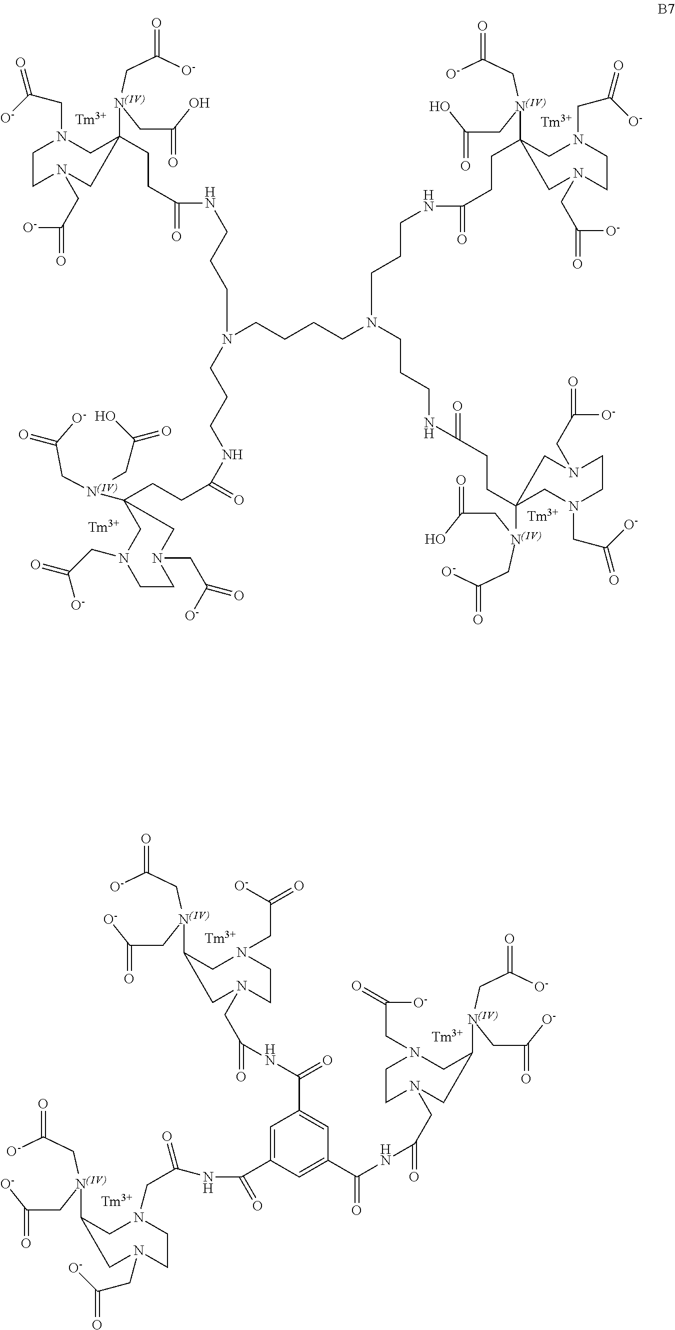

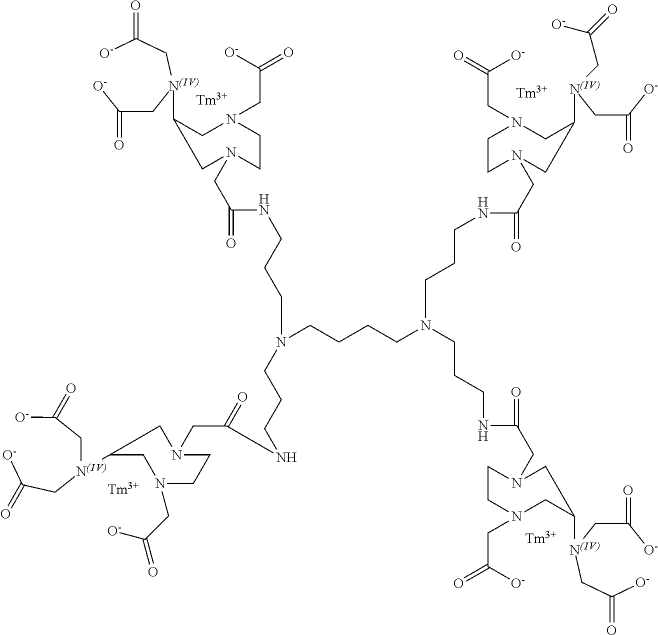

The molecules above are illustrating not limiting examples; for example AAZTA ionic and non ionic mulimers are illustrated.

Depending on embodiments, the encapsulating systems of the application, incorporating q≧2 chelates in the form of monomers q≧2 or of multimers of monomers q≧2, comprise, on the one hand, chelates that are encapsulated and free inside the compartment of the encapsulating system and, on the other hand, in addition chelates associated with the membrane (denoted membrane chelates), for example by virtue of lipophilic groups. Such membrane chelates are described in detail in document WO 2006032705, with the example of chelates of PCTA type, in particular. In embodiments, the membrane chelates are oriented (for the chelate part) essentially toward the inside of the system, i.e. the chelates are predominantly oriented toward the inside (the polar part comprising the polynitrogenous nucleus is located at the inner surface of the system, i.e. inside the liposome). In embodiments, the membrane chelates are oriented (for the chelate part) essentially toward the outside of the system (the lipophilic chelate then comprises a lipophilic part for association with the membrane, and a polar part comprising the polynitrogenous nucleus and located at the outer surface of the system). There will thus, for example, be provided liposomes comprising, on the one hand, encapsulated monomeric q≧2 chelates free inside, and on the other hand, membrane chelates oriented toward the inside and/or toward the outside of the liposome.

It is clear that the chemical shift Delta of the applicant's compounds are advantageously:
of at least 7 ppm for spherical (non-deformed) systems using essentially chelates in the form of monomeric q≧2 chelates;
of at least 10 (preferably at least 12) ppm for spherical non-deformed systems using essentially chelates in the form of dimers of monomeric q>2 chelates;
of at least 15 (preferably at least 20) for non-deformed systems using essentially chelates in the form of trimers of monomeric q≧2 chelates, of at least 20 (preferably at least 25) for systems using essentially chelates in the form of tetramers of monomeric q≧2 chelates.

Depending on embodiments, the encapsulating systems of the application, incorporating monomeric q≧2 chelates or multimers of monomeric q≧2 chelates, have a structure that is modified so as to improve the signal. It is in particular the susceptibility effect, and is advantageously a nonspherical deformed liposome (termed shrunken) described in Chem. Commun., 2008, 600-602 and WO 2006/095234, obtained by applying an osmotic shock and using suitable membrane constituents. To obtain these liposomes, use will advantageously be made of the protocol described in the prior art (incorporated by way of reference), for example, in Angewandte Chemie, vol. 46, issue 6, p 807-989 and Chem. commun, 2008, 600-602. For these nonspherical systems, use will be made of free monomeric q≧2 chelates or multimers of monomeric q≧2 chelates, with lipophilic lanthanide complexes (having q=1 or 2) inserting into the membrane in order to control the orientation, in the magnetic field, of the nonspherical liposome. Use may be made (nonpreferred variant) of deformed systems which increase these values, but on the condition that they are sufficiently stable. In these deformed (non-spherical) ES, the chemical shift Delta of the applicant's compounds are advantageously:
of at least 20 ppm, preferably at least 25 ppm, for deformed (non-spherical) systems using essentially chelates in the form of monomeric q≧2 chelates;
of at least 40 ppm, preferably at least 50 ppm, for deformed (non-spherical) systems using essentially chelates in the form of dimers of monomeric q≧2 chelates;
   leading thus to a signal efficiency (Delta per monomer) R≧20, preferably R≧25, (instead of R of about 10 to 15 in the prior art).

It is precised that the method of measure of Delta value is known by the one skilled in the art and illustrated in the detailed examples and in Chem.Commun, 2008, 600-602 and references thereof (Z-spectrum spectra typically at 310 K and 300 MHz).

Depending on embodiments, the encapsulating systems (preferably spherical) of the application, incorporating monomeric q≧2 chelates or multimers of monomeric q≧2 chelates, also comprise at least one biovector for targeting a pathological region of diagnostic interest, the biovector being advantageously an amino acid, a peptide, a polypeptide (preferably of less than 20 amino acids, notably of 4 to 10 amino acids), a vitamin, a monosaccharide or polysaccharide, an antibody or a nucleic acid, advantageously a peptide or a polypeptide, in particular a biovector targeting cell receptors (in particular all the receptors described below), a pharmacophor (organic molecule with pharmacological activity), an angiogenesis-targeting biovector, an MMP-targeting biovector, a tyrosine-kinase-targeting peptide, an atheroma-plaque-targeting peptide or an amyloid-plaque-targeting biovector.

Advantageously, in the context of the present invention, the term “biovector” is intended to mean any biomolecule capable of specifically targeting a biological target such as a cell receptor, or a tissue component, for example chosen from myocardial cells, endothelial cells, epithelial cells or tumor cells, or cells of the immune system or the components of normal or pathological tissue architecture.

More broadly, the biovector(s) is (are), for example, chosen from the following list (the documents and references between parentheses are examples and not a limiting list):

1) Biovectors targeting VEGF receptors and angiopoietin (described in WO 01/97850), polymers such as polyhistidine (U.S. Pat. No. 6,372,194), fibrin-targeting polypeptides (WO 2001/9188), integrin-targeting peptides (WO 01/77145, WO 02/26776 for av(33, WO 02/081497, for example RGDWXE), pseudopeptides and peptides for targeting metalloproteases MMP (WO 03/062198, WO 01/60416), peptides targeting, for example, the KDR/Flk-1 receptor or Tie-1 and 2 receptors (WO 99/40947, for example), sialyl Lewis glycosides (WO 02/062810 and Müller et al, Eur. J. Org. Chem., 2002, 3966-3973), antioxidants such as ascorbic acid (WO 02/40060), tuftsin-targeting biovectors (for example, U.S. Pat. No. 6,524,554), biovectors for targeting G protein receptors, GPCRs, in particular cholecystokinin (WO 02/094873), associations between an integrin antagonist and a guanidine mimic (U.S. Pat. No. 6,489,333), αvβ3-targeting or αvβ5-targeting quinolones (U.S. Pat. No. 6,511,648), benzodiazepines and analogs targeting integrins (US A 2002/0106325, WO 01/97861), imidazoles and analogs (WO 01/98294), RGD peptides (WO 01/10450), antibodies or antibody fragments (FGF, TGFβ, GV39, GV97, ELAM, VCAM, which are TNF- or IL-inducible (U.S. Pat. No. 6,261,535)), targeting molecules which are modified by interaction with the target (U.S. Pat. No. 5,707,605), agents for targeting amyloid deposits (WO 02/28441, for example), cleaved cathepsin peptides (WO 02/056670), mitoxantrones or quinones (U.S. Pat. No. 6,410,695), epithelial-cell-targeting polypeptides (U.S. Pat. No. 6,391,280), cysteine protease inhibitors (WO 99/54317), the biovectors described in: U.S. Pat. No. 6,491,893 (GCSF), US 2002/0128553, WO 02/054088, WO 02/32292, WO 02/38546, WO 20036059, U.S. Pat. No. 6,534,038, WO 0177102, EP 1 121 377, Pharmacological Reviews (52, No. 2, 179: growth factors PDGF, EGF, FGF, etc.), Topics in Current Chemistry (222, W. Krause, Springer), Bioorganic & Medicinal Chemistry (11, 2003, 1319-1341; αvβ3-targeting tetrahydrobenzazepinone derivatives).

2) Angiogenesis inhibitors, in particular those tested in clinical trials or already marketed, in particular:
inhibitors of angiogenesis involving FGFR or VEGFR receptors, such as SU101, SU5416, SU6668, ZD4190, PTK787, ZK225846, azacyclic compounds (WO 00244156, WO 02059110);
inhibitors of angiogenesis involving MMPs, such as BB25-16 (marimastat), AG3340 (prinomastat), solimastat, BAY12-9566, BMS275291, metastat, neovastat;
inhibitors of angiogenesis involving integrins, such as SM256, SG545, adhesion molecules which block EC-ECM (such as EMD 121-974, or vitaxin);
medicaments with a more indirect mechanism of antiangiogenic action, such as carboxyamidotriazole, TNP470, squalamine, ZD0101;
the inhibitors described in document WO 99/40947, monoclonal antibodies which are very selective for binding to the KDR receptor, somatostatin analogs (WO 94/00489), selectin-binding peptides (WO 94/05269), growth factors (VEGF, EGF, PDGF, TNF, MCSF, interleukins); VEGF-targeting biovectors described in Nuclear Medicine Communications, 1999, 20;
the inhibitory peptides of document WO 02/066512.

3) Biovectors capable of targeting receptors: CD36, EPAS-1, ARNT, NHE3, Tie-1, 1/KDR, Flt-1, Tek, neuropilin-1, endoglin, pleiotropin, endosialin, Axl., alPi, a2ss1, a4P1, a5 μl, eph B4 (ephrin), the laminin A receptor, the neutrophilin receptor 65, the leptin receptor OB-RP, the chemokine receptor CXCR-4 (and other receptors mentioned in document WO 99/40947), LHRH, bombesin/GRP, receptors for gastrin, VIP, CCK.

4) Biovectors of tyrosine kinase inhibitor type.

5) Known GPIIb/IIIa receptor inhibitors, chosen from: (1) the fab fragment of a monoclonal antibody against the GPIIb/IIIa receptor, Abciximab, (2) small peptide and peptidomimetic molecules injected intravenously, such as eptifibatide and tirofiban.

6) Fibrinogen receptor antagonist peptides (EP 425 212), IIb/IIIa receptor ligand peptides, fibrinogen ligands, thrombin ligands, peptides capable of targeting atheroma plaque, platelets, fibrin, hirudin-based peptides, guanine-based derivatives targeting the IIb/IIIa receptor.

7) Other bioovectors or biologically active fragments of biovectors known to those skilled in the art as medicaments, having an anti-thrombotic, anti-platelet-aggregation, anti-atherosclerotic, anti-restenoic or anticoagulant action.

8) Other biovectors or biologically active fragments of biovectors targeting αvβ3, described in association with DOTAs in patent U.S. Pat. No. 6,537,520, chosen from the following: mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, leutinizing hormone releasing factor.

9) Certain biovectors targeting particular types of cancers, for example peptides targeting the ST receptor associated with colorectal cancer, or the tachykinin receptor.

10) Biovectors using phosphine-type compounds.

11) The biovectors for targeting P-selectin, E-selectin; for example, the 8-amino-acid peptide described by Morikawa et al, 1996, 951, and also various sugars.

12) Annexin V or biovectors targeting apoptotic processes.

13) Any peptide obtained by targeting technologies, such as phage display, optionally modified with unnatural amino acids (http//chemlibrary.bri.nrc.ca), for example peptides derived from phage display libraries: RGD, NGR, KGD, RGD-4C.

14) Other peptide biovectors known for targeting atheroma plaques, mentioned in particular in document WO 2003/014145.

15) Vitamins, in particular folic acid (folic acid, dideaza compounds) and its known derivatives capable of targeting folate receptors.

16) Ligands for hormone receptors, including hormones and steroids.

17) Opioid-receptor-targeting biovectors.

18) TKI-receptor-targeting biovectors.

19) LB4 and VnR antagonists.

20) Nitroimidazole and benzylguanidine compounds.

21) Biovectors summarized in Topics in Current Chemistry, vol. 222, 260-274, Fundamentals of Receptor-based Diagnostic Metallopharmaceuticals, in particular:
biovectors for targeting peptide receptors overexpressed in tumors (LHRH receptors, bombesin/GRP, VIP receptors, CCK receptors, tachykinin receptors, for example), in particular analogs of somatostatin or of bombesin, optionally glycosylated octreotide peptide derivatives, VIP peptides, alpha-MSHs, CCK-B peptides;
peptides chosen from: RGD cyclic peptides, fibrin-targeting peptides, tuftsin-targeting peptides, peptides for receptor targeting: laminin.

22) Oligosaccharides, polysaccharides and derivatives of monosaccharides, derivatives targeting Glut receptors (monosaccharide receptors) or glutamine transporters.

23) Biovectors used for smart-type products.

24) Myocardial viability markers (for example, tetrofosmin and hexakis(2-methoxy-2-methylpropylisonitrile)).

25) Sugar and fat metabolism traces.

26) Ligands of neurotransmitter receptors (D, 5HT, Ach, GABA, NA, NMDA receptors).

27) Oligonucleotides.

28) Tissue factor.

29) Biovectors described in WO 03/20701, in particular the PK11195 ligand for the peripheral benzodiazepine receptor.

30) Fibrin-binding peptides, in particular the peptide sequences described in WO 03/11115.

31) Amyloid plaque aggregation inhibitors described for instance in WO 02/085903.

32) Compounds for targeting Alzheimer's disease, in particular compounds comprising backbones of benzothiazole, benzofuran, styrylbenzoxazole/thiazole/imidazole/quinoline, styrylpyridine or stilbene type, and known derivatives thereof.

These biovectors attached to the encapsulating system (typically at the external surface, where appropriate, by means of lipophilic groups for anchoring in the membrane) make it possible to reach the target region thus recognized, specifically. Patent WO 2006032705 illustrates numerous examples of chemical coupling, incorporated by way of reference, of the liposome with various categories of biovectors, and numerous examples of compositions of liposomes, incorporated by way of reference. Advantageously the ES (and notably the liposomes) forming lipids comprise phospholipids or hydrogenated phospholipids or derivatives thereof among phosphatidylcholines (lecithins) (PC), phosphatidylethanolamines (PE), lysolecithins, lysophosphatidylethanolamines, phosphatidylserines (PS), phosphatidylglycerols (PG), phosphatidylinositol (PI), sphingomyelins, cardiolipin, phosphatidic acids (PA), fatty acids, gangliosides, glucolipids, glycolipids, mono-, di or triglycerides, ceramides or cerebrosides. Advantageously, a mixture of saturated and unsaturated phospholipids and of cholesterol is used, notably in the proportion 40/10/50 to 60/5/35 for instance 55/5/40. The biovector is used preferably as 0.5 to 10% of the constituents, notably 3, 5, 7%.

For instance, a lipid solution containing 55 mol % POPC, 5% DPPG, 34% cholesterol, 5% DSPE-PEG₂₀₀₀ and 1% of the biovector (biovector coupled to a lipophilic anchoring group) is used for preparing the encapsulating ES system.

Where appropriate, the encapsulating system also comprises, for example inside the liposome, a suitable therapeutic agent for treating the diseased region to be treated.

Depending on embodiments, systems of the application will be used for targeting cells using T2 imaging properties of these systems associated with susceptibility effects.

The applicant has described most particularly liposome-type CEST agents encapsulating chelates which are free inside the liposome. Patent WO 2006032705 also describes lipid systems of nanoparticle type, and in particular emulsions (also denoted emulcest), with or without fluoro compounds of perfluorocarbon type, for which chelates are grafted onto the outer surface, using emulsions (also called nanodroplets, described in particular in U.S. Pat. No. 6,676,963). The applicant has also studied the grafting of monomeric q≧2 chelates onto the outer face of nanoemulsions and of micelles (lipid monolayer with a polar part facing the outside of the micelle). In these particulate systems, the CEST effect due mainly to the chelates grafted onto the outer face (and typically inserted partly into the lipid layer by means of lipophilic groups of the chelates and, where appropriate, of aliphatic and/or aromatic linker groups) is obtained by virtue of the very large number of chelates grafted to the particles. Depending on embodiments, the encapsulating system is thus an emulsion or a micelle (not an inverse micelle), which is advantageously perfluorinated, the chelates being essentially located on the outer face of the system.

The applicant has thus studied the grafting of q≧2 chelates to lipid nanoparticles described in WO 2008/132666 and WO 2007/141767.

The invention is also advantageous for the grafting of monomeric q≧2 chelates to compounds of the type such as polymers used in medical imaging, dendrimers, polypeptide or protein systems, polysaccharides, nucleic acids, polymeric nanoparticles, metal nanoparticles, in particular nanoparticles of metal oxides, including lanthanides (nanoparticles, the metal or the mixture of metals of which does not interfere in such a way as to impede the CEST effect of the chelates).

According to an other aspect, the invention relates to the use of a composition described in the application, and in particular of a composition comprising an encapsulating system encapsulating at least one CEST agent constituted of a monomeric chelate of a chelate of q≧2 type, or of a multimer of monomeric chelates of q≧2 type, said chelate being free inside the encapsulating system:
for CEST imaging
for the preparation of a diagnostic agent for in vivo CEST imaging.

To produce the contrast media compositions of the invention, the liposomes are formulated in pharmaceutically physiologically tolerable liquid carrier medium, e.g. an aqueous solution which may include one or more additives, such as pH modifying agents, chelating agents, antioxidants, tonicity modifying agents, cryoprotectants, further contrast agents, etc. Pharmaceutically acceptable formulations are prepared as known in the art of lipocest agents and reminded in WO2006032705 notably.

The compounds of the invention are administered at advantageously low concentrations, typically 1 to 100 nM of ES (notably liposomes), preferably 30 to 100 nM as detailed in the examples. It is precised that even by increasing the concentration of the ES injected with q=1, the Delta shift would not significantly change (an increase of concentration of ES injected allows to improve the sensibility for a certain Delta shift value, but not the Delta value). With monomeric q=2 chelates or multimers thereof the concentration of ES injected to the patient is for instance 50 nM, corresponding to a lanthanide concentration (Tm for instance) of about 10 to 20 mM. Inside the ES spherical system (liposome notably), the concentration of the trimers of monomeric q=2 chelates will be advantageously about 0.3M or more, corresponding to 0.9 M or more of the lanthanide (the trimer contains 3 lanthanide, for example Tm), allowing to reach a Delta advantageously of about 30 ppm for spherical systems. Whereas the concentration of the monomeric q=1 chelates inside spherical liposomes was typically 0.2 M, leading to about 0.2 M lanthanide and a shift of 4 ppm.

The invention also relates to the use of a composition comprising an encapsulating system ES encapsulating at least one shift agent wherein the at least one shift agent is constituted of a monomeric chelate of a chelate of q≧2 type, or of a multimer of monomeric chelates of q≧2 type, and wherein said chelate is free inside the encapsulating system, for the preparation of a diagnostic agent for Cest imaging.

In particular, said method of Cest imaging does not comprise a step of administration of said composition.

The description of detailed examples which follows makes it possible to illustrate the invention.

### DESCRIPTION OF THE FIGURES

FIG. 1 represents the standardized intensity (standardized relative to the value obtained during irridation at +20 ppm) as a function of the frequency of irradiation for a spherical liposome encapsulating the PCTA-Tm q=2 chelate according to the invention in CEST imaging.

FIG. 2 represents Is/Io (%) for a spherical liposome encapsulating the PCTA-Tm chelate according to the invention in CEST imaging, where Is is the intensity measured at the time of saturation on the left of the external water peak, and Io is the intensity at the time of saturation on the right of the peak, as a function of the frequency of irradiation.

FIG. 3 represents the standardized intensity (standardized relative to the value obtained during irradiation at +20 ppm) as a function of the frequency of irradiation for a spherical liposome encapsulating the DO3A-Tm q=2 chelate according to the invention, in CEST imaging.

FIG. 4 represents Is/Io (%) for a spherical liposome encapsulating the DO3A-Tm chelate according to the invention in CEST imaging

FIG. 5a represents the schematic structures of the chelates and the atom names for DOTA (chelate q=1) in exchange with one water molecule H2O WC, and for DO3A (chelate q=2) in exchange with the two water molecules H2O WA and H2O WB FIG. 5b represents angular projections of the hydrated faces of the monomeric chelates DOTA (q=1) and DO3A (q=2).

FIG. 5c represents the structure of a chelate with the favorable situation of water molecule in the cone for shift effect.

FIG. 6 represents a schematic view of the positions for a monomeric chelate q=1

FIG. 7 represents a schematic view of the positions for a dimeric chelate made of two chelates q=1.

FIG. 8 represents a schematic view of the positions for a monomeric chelate q=2 expected from the prior art.

FIG. 9 represents a schematic view of the positions for a monomeric chelate q=2 of the applicant.

### PART I/SYNTHESIS OF THE MONOMERIC q=2 COMPLEXES (CHELATE+LANTHANIDE)

### Examples 1 to 22

The protocols are presented for Tm³⁺as metal. However, those skilled in the art, by virtue of their general knowledge, are able to produce these compounds with other metals that can be used in CEST.

### Example 1

### Compound A

### PCTA Derivative Chelate

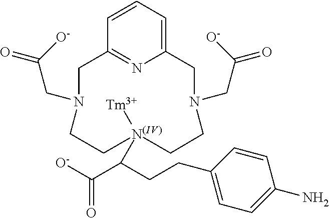

The synthesis is identical to that described in example 1 of patent WO 2006/100305, the complexation being carried out (stage i) with 705 mg of TmCl₃. 6H₂O C₂₅H₃₀N₅O₆Tm

m/z (ES−)=664.

### Example 2

### Compound B

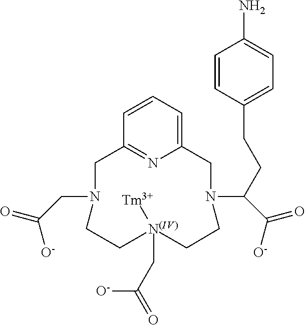

The synthesis is identical to that described in example 2 of patent WO 2006/100305, the complexation being carried out (stage d) with 1.4 g of TmCl₃. 6H₂O C₂₅H₃₀N₅O₆Tm

m/z (ES−)=664.

### Example 3

### Compound C

### DO3A Derivative Chelate

a)

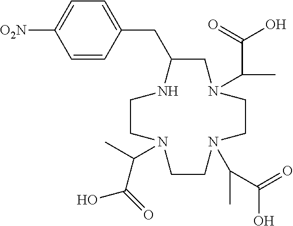

130 mg (0.23 mmol) of trimethyl a,a′,a″-trimethyl-(2S)-2-(4-nitrobenzyl)]-1,4,7,10-tetraazacyclododecane-4,7,10-triacetate the synthesis of which is described by Woods et al (Dalton Trans), 2005, 3829-3837, are dissolved in 5 ml of THF. 1.8 ml of 1M sodium hydroxide and 5 ml of water are added. The reaction medium is stirred at 55° C. for 24 h before being evaporated to dryness. The product is taken up in water and purified by preparative HPLC. 100 mg are obtained.

m/z (ES+)=524.

b)

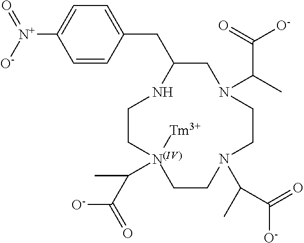

100 mg of the intermediate obtained in a) are dissolved in 4 ml of water. The pH of the solution is brought to 5 by adding 2N sodium hydroxide. After the addition of 71.3 mg of TmCl₃. 6H₂O, the reaction medium is heated at 80° C. for 6 h while maintaining the pH at 5 by adding 2N sodium hydroxide. After filtration, the residue is crystallized from ethanol. The precipitate is dissolved in water and treated with a Chelex® 100 resin (Bio-Rad). After filtration and precipitation from ethanol, the precipitate is filtered off and dried. m=120 mg.

m/z (ES⁻)=688.

### c) Reduction of the Nitro Group NO₂ to NH₂

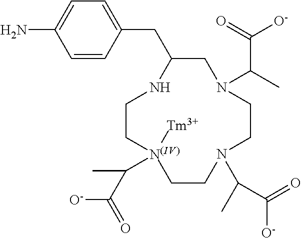

Starting from 120 mg of the compound obtained in b), applying the same procedure as that described in stage j of example 1 of patent WO 2006/100305, 100 mg of compound C are obtained.

m/z (ES−)=658.

### Example 4

### Compound D

a)

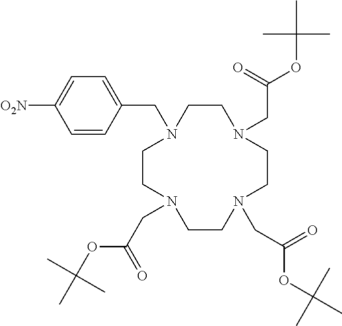

25 g (49 mmol) of the intermediate obtained in a) (ex29) are suspended in 175 ml of CH₃CN with 13.5 g (98 mmol) of K₂CO₃, under argon. 1.16 eq (12.2 g) of 4-nitrobenzylbromide are diluted in 65 ml of CH₃CN, and then added dropwise. The reaction medium is left to stir at reflux for 24 h and is then evaporated to dryness. The product is taken up with 250 ml of CH₂Cl₂, and washed 3 times with 150 ml of H₂O. The organic phase is concentrated and then taken up with 250 ml of 1N HCl and washed with 250 ml of ethyl ether. The aqueous phase is basified (pH 9) with Na₂CO₃. The product is extracted with 250 ml of CH₂Cl₂ and dried over MgSO₄. The organic phase is dried under vacuum. m(crude)=26 g, which are purified on 1 kg of silica.

Eluent: 90/10 (CH₂Cl₂/MeOH)

m/z (ES+)=650.

b)

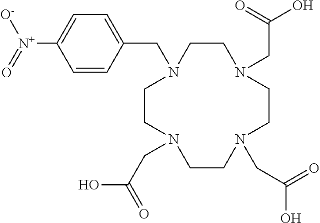

Starting from 18 g of the compound obtained in a), applying the same procedure as that described in stage a) for compound B, 10 g of the compound are obtained.

m/z (ES+)=482.

c)

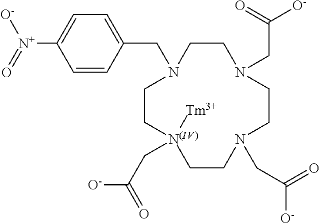

Starting from 13 g of the compound obtained in b), applying the same procedure as that described in stage b) for compound C, 10 g of the compound are obtained.

m/z (ES+)=646.

### d) Reduction of the Nitro

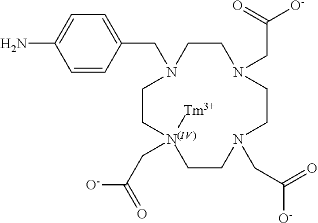

Starting from 13 g of the compound obtained in c), applying the same procedure as that described in stage j of example 1 of patent WO 2006/100305, 12 g of compound D are obtained.

m/z (ES−)=616.

### Example 5

### Compound E

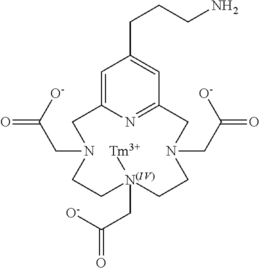

The synthesis is identical to that described in example 11 of patent WO 2006/100305, the complexation being carried out (stage e) with TmCl₃. 6H₂O C₂₀H₂₈N₅O₆Tm

m/z(ES−)=602.

### Example 6

### Compound F

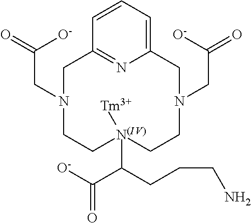

The synthesis is identical to that described in example 13 of patent WO 2006/100305, the complexation being carried out (stage c) with TmCl₃. 6H₂O

C₂₀H₂₈N₅O₆Tm

m/z(ES−)=602.

### Example 7

### Compound G

a)

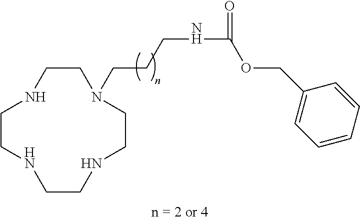

1 g of compound 18b described by S. J. Krivickas et al; JOC, 2007, 72, pp 8280-8289 are dissolved in 10 ml of TFA. The medium is stirred for 1 h and then evaporated to dryness. The product is taken up in ether and then filtered. 550 mg of the compound are obtained.

m/z (ES+)=406 (n=4).

b)

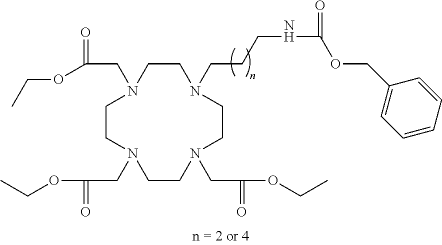

550 mg of the compound obtained in a) and 618 mg of K₂CO₃ are dissolved in 10 ml of acetonitrile.

747 mg of ethyl bromoacetate (3.3 equiv) are added dropwise and then the mixture is stirred at 60° C. for 4 days. After evaporation to dryness, the product is taken up in 20 ml of dichloromethane and 4 ml of water. After separation of the phases, the organic phase is evaporated and purified by flash chromatography on silica with 2% of methanol in dichloromethane. 2 g of the compound are obtained.

m/z (ES+)=749 (n=4).

c)

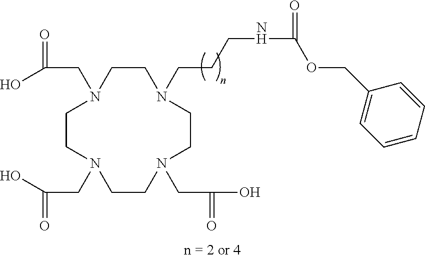

Starting from 2 g of the compound obtained in b), applying the same procedure as that described in stage a) of example 3, 1.2 g of the compound are obtained.

m/z (ES+)=580 (n=4).

### d) Complexation

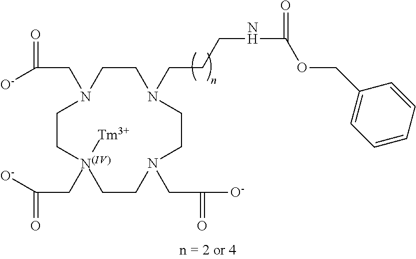

Starting from 1.2 g of the compound obtained in c), applying the same procedure as that described in stage b) of example 3, 1.4 g of the compound are obtained.

m/z (ES−)=744 (n=4).

e)

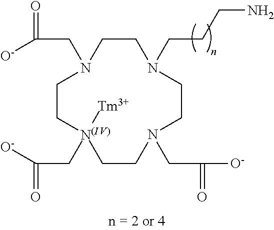

Starting from 1.4 g of the compound obtained in d), applying the same procedure as that described in stage b) of example 13 of patent WO 2006/100305, 1 g of compound G is obtained.

m/z (ES−)=610 (n=4).

### Example 8

### Compound H

a)

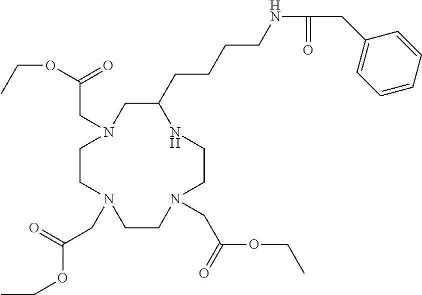

By applying the same procedure as that described in stage b) of example 7, 380 mg are obtained starting from 250 mg of compound 15 described by P. L. Cox in J. Chem. Soc. Perkin Trans.1, 1990, p 2567.

m/z=620 (ES+).

b)

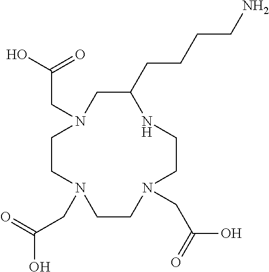

Starting from 350 mg of the compound obtained in a), applying the same procedure as that described in stage a) of example 3, 200 mg of the compound are obtained.

m/z=418 (ES+).

c)

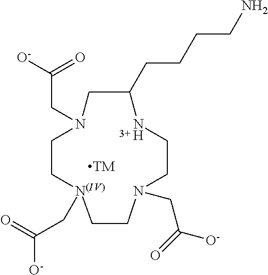

Starting from 200 mg of the compound obtained in b), applying the same procedure as that described in stage b) of example 3, 250 mg of compound H are obtained.

m/z=582 (ES−).

### Example 9

### Compound I

### HOPO Chelate

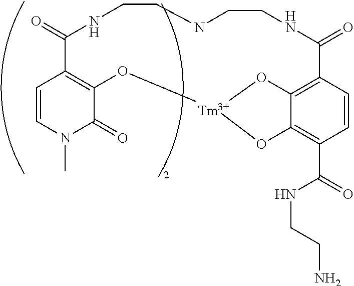

Synthesis described in Angew. Chem. Int. Ed, 2008, 47, pp 8568, the complexation being carried out with TmCl₃.6H₂O C₃₀H₃₅N₈O₁₀Tm

m/z(ES−)=835.

### Example 10

### Compound J

### AAZTA Chelate

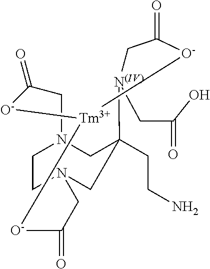

Obtained by carrying out the complexation with TmCl₃. 6H₂O starting from the ligand of which the synthesis is described in WO 2006/100305,

C₁₅H₂₃N₄O₈Tm

m/z(ES−)=555.

### Example 11

### a) Compound K

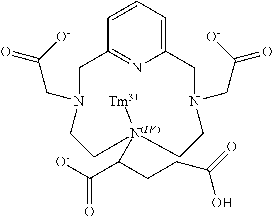

The synthesis is identical to that described in example 3 of patent WO 2006/100305, the complexation being carried out (stage d) with TmCl₃. 6H₂O

C₂₀H₂₅N₄O₈Tm

m/z(ES−)=617.

### b) Compound K′

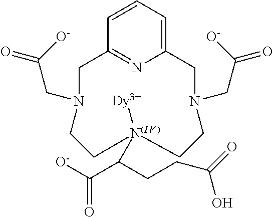

The synthesis is identical to that described in example 3 of patent WO 2006/100305, the complexation being carried out (stage d) with DyCl₃. 6H₂O

C₂₀H₂₅N₄O₈Dy

m/z(ES−)=611.

### Example 12

### Compound L

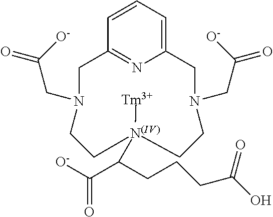

The synthesis is identical to that described in example 15 of patent WO 2006/100305, the complexation being carried out (stage d) with TmCl₃. 6H₂O

C₂₁H₂₇N₄O₈Tm

m/z(ES−)=631.

### Example 13

### Compound M

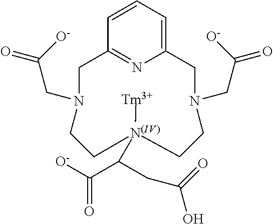

The synthesis is identical to that described in example 16 of patent WO 2006/100305, the complexation being carried out (stage d) with TmCl₃. 6H₂O C₁₉H₂₃N₄O₈Tm

m/z(ES−)=603.

### Example 14

### Compound N

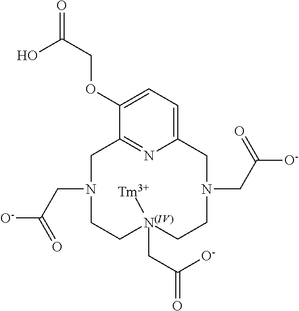

The synthesis is identical to that described in example 4 of patent WO 2006/100305, the complexation being carried out (stage j) with TmCl₃. 6H₂O C₁₉H₂₃N₄O₉Tm

m/z(ES−)=619.

### Example 15

### Compound O

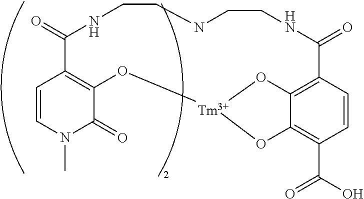

Obtained by carrying out the complexation with TmCl₃. 6H₂O starting from the ligand of which the synthesis is described in Angew. Chem. Int. Ed, 2008, 47, pp 8568. C₂₈H₂₉N₆O₁₁Tm

m/z(ES−)=793.

### Example 16

### Compound P

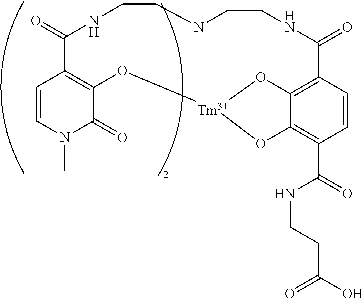

Obtained by carrying out the complexation with TmCl₃. 6H₂O starting from the ligand of which the synthesis is described in Angew. Chem. Int. Ed, 2008, 47, pp 8568. C₃₁H₃₄N₇O₁₂Tm

m/z(ES−)=864.

### Example 17

### Compound Q

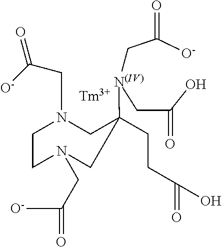

Obtained by carrying out the complexation with TmCl₃. 6H₂O starting from the ligand of which the synthesis is described in PCT/EP2006/063368.

C₁₆H₂₂N₃O₁₀Tm

m/z(ES−)=584.

### Example 18

### Compound R

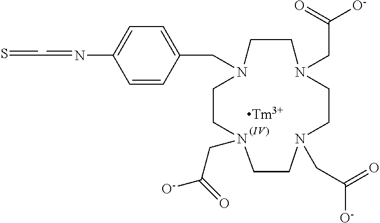

2 g of intermediate obtained in stage d) for compound D are dissolved in a mixture constituted of 16 ml of CHCl₃ and 24 ml of H₂O. 0.5 ml of thiophosgene is added dropwise. The reaction medium is stirred for 4 h at ambient temperature. The aqueous phase is washed three times with CHCl₃ and is then evaporated under vacuum, the temperature being maintained below 35° C. The product is taken up in ether and filtered. 2 g of compound R are obtained.

C₂₂H₂₈N₅O₆STm

m/z(ES−)=658.

### Example 19

### Compound S

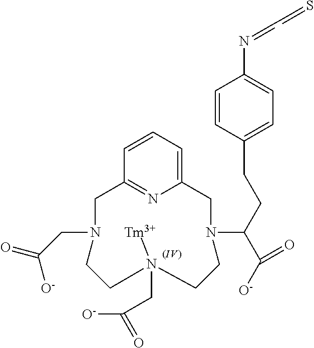

The synthesis is identical to that described in example 9 of patent WO 2006/100305, the complexation being carried out with TmCl₃. 6H₂O.

C₂₆H₂₈N₅O₆STm

m/z(ES−)=706.

### Example 20

### Compound T

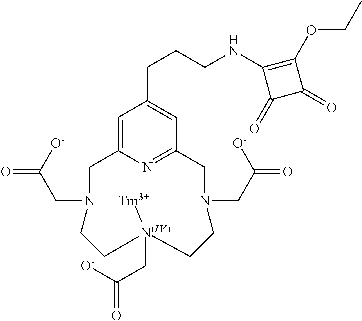

The synthesis is identical to that described in example 12 of patent WO 2006/100305, the complexation being carried out with TmCl₃. 6H₂O.

C₂₆H₃₂N₅O₉Tm

m/z(ES−)=726.

### Example 21

### Compound U

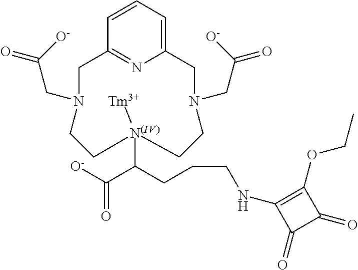

The synthesis is identical to that described in example 14 of patent WO 2006/100305, the complexation being carried out with TmCl₃. 6H₂O.

C₂₆H₃₂N₅O₉Tm

m/z(ES−)=726.

### Example 22

### Compound V

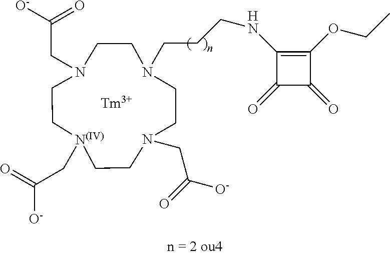

Starting from 1 g of the compound obtained in e) for compound G, applying the same procedure as that described in stage a) of example 12 of patent WO 2006/100305, 1.1 g of compound V are obtained.

C₂₆H₄₀N₅O₉Tm

m/z (ES−)=734 (n=4).

### PART II/PREPARATION OF COMPOUNDS DENOTED CORE (NUCLEUS) FOR THE SYNTHESIS OF MULTIMERS OF MONOMERIC CHELATES a≧2 NOTABLY a=2

| | R′(COOH)ₙ | R″(NH₂)ₙ |
|---|---|---|
| n = 2: Dimer | 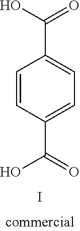 | Ethylene diamine VI |
| n = 3: Trimer | 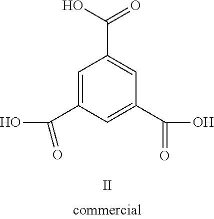 | 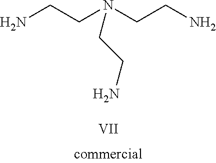 |
| | | 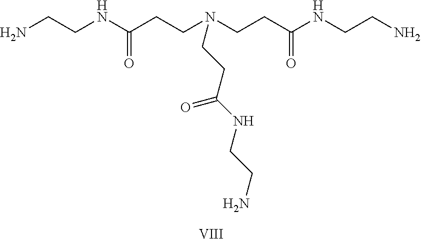 |
| n = 4: Tetramer | 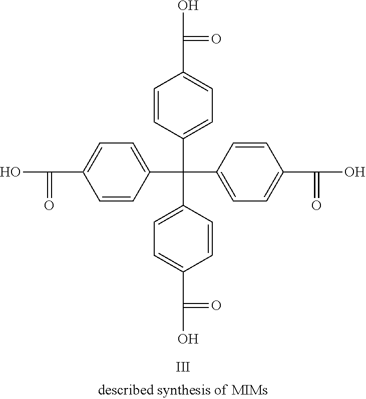 | 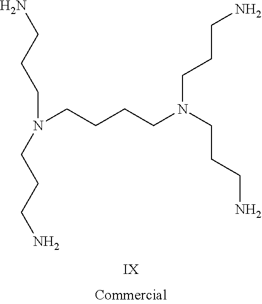 |
| | 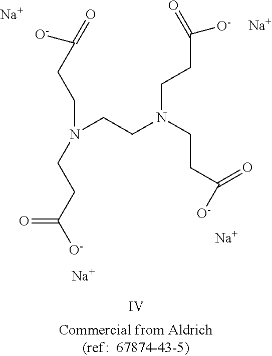 | 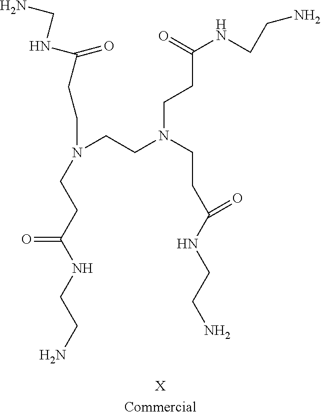 |
| n = 6: Hexamer | 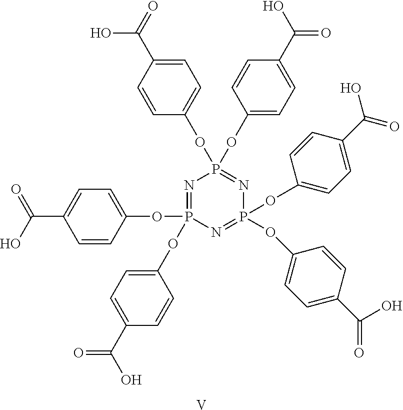 | |

### PART III/SYNTHESIS OF MULTIMERS OF MONOMERIC CHELATES q≧2 NOTABLY q=2

### Example 23

### Condensation of Aromatic Amines (R1-NH) with Cyanuric Chloride

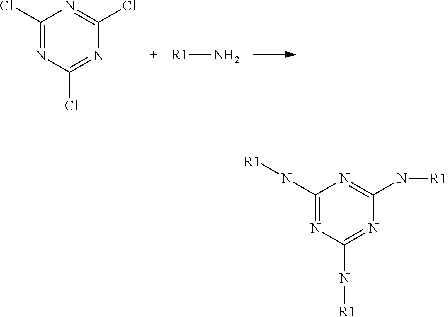

0.19 g (1 mmol) of cyanuric chloride, dissolved in 10 ml of dioxane, is introduced, in a single portion, into 50 ml of an aqueous solution obtained by dissolution of x g (3.3 mmol, 3.3 eq) of aminated compound (R1-NH₂) and 456 g (3.3 eq) of K₂CO₃. The pH is maintained at 9 by adding K₂CO₃. After 18H at ambient temperature, the pH is brought back to 6.5 by adding IRC50 resin. The reaction medium is filtered and concentrated, before being poured dropwise into ethanol. The precipitate formed is filtered off, and washed with ethanol and then ether. The product is purified by preparative HPLC on a Lichrospher RP18, 15 μm, 300 A, 400×60 mm column with a TFA/CH₃CN mobile phase.

| | Compound |
|---|---|
| R1-NH₂ x (g) | obtained |
| 2.2 g of compound A | C₇₈H₈₇N₁₈O₁₈Tm₃ |
| | m/z (ES-) = 2069 |
| 2.2 g of compound B | C₇₈H₈₇N₁₈O₁₈Tm₃ |
| | m/z (ES-) = 2069 |
| 2.2 g of compound C | C₇₅H₁₀₅N₁₈O₁₈Tm₃ |
| | m/z (ES-) = 2052 |
| 2.0 g of compound D | C₆₆H₈₇N₁₈O₁₈Tm₃ |
| | m/z (ES-) = 1925 |

### Example 24

### Condensation of Aliphatic Amines (R2-NH₂) on Polyacid Cores R′(COOH)ₙ

R′(COOH)ₙ+nR2-NH₂→R′(CONH—R2)ₙ

0.8 mmol of the acid compound and 1.1 equivalent per acid function of the amine compound (R2-NH₂) are dissolved by heating to 40° C. in 15 ml of DMAC. After dissolution, 153 mg (0.8 mmol per acid function) of EDCI, 19 mg (0.14 mmol) of HOBT and 0.15 ml of TEA are added. The mixture is left at 45° C. for 18H. The reaction medium is cooled, before being poured dropwise into ethanol. The precipitate obtained is filtered off and washed with ether.

The product is purified by ultrafiltration through a 1 KD membrane, or by preparative HPLC.

Compounds obtained with R2-NH₂=compound E, F, G, H, I or J:

| | I | II | III | IV | V |
|---|---|---|---|---|---|
| E | C₄₈H₅₈N₁₀O₁₄Tm₂ | C₆₉H₈₄N₁₅O₂₁Tm₃ | C₁₀₉H₁₂₄N₂₀O₂₈Tm₄ | C₉₄H₁₂₈N₂₂O₂₈Tm₄ | C₁₆₂H₁₈₆N₃₃O₄₈P₃Tm₆ |
| | m/z(ES-) = 1336 | m/z(ES-) = 1964 | m/z(ES-) = 1417 | m/z(ES-) = 1343 | m/z(ES-) = 1488 (z = 3) |
| | | | (z = 2) | (z = 2) | |
| F | C₄₈H₅₈N₁₀O₁₄Tm₂ | C₆₉H₈₄N₁₅O₂₁Tm₃ | C₁₀₉H₁₂₄N₂₀O₂₈Tm₄ | C₉₄H₁₂₈N₂₂O₂₈Tm₄ | C₁₆₂H₁₈₆N₃₃O₄₈P₃Tm₆ |
| | m/z(ES-) = 1336 | m/z(ES-) = 1964 | m/z(ES-) = 1417 | m/z(ES-) = 1343 | m/z(ES-) = 1488 (z = 3) |
| | | | (z = 2) | (z = 2) | |
| G | C₄₈H₇₄N₁₀O₁₄Tm₂ | C₆₉H₁₀₈N₁₅O₂₁Tm₃ | C₁₀₉H₁₅₆N₂₀O₂₈Tm₄ | C₉₄H₁₆₀N₂₂O₂₈Tm₄ | C₁₆₂H₂₃₄N₃₃O₄₈P₃Tm₆ |
| | m/z(ES-) = 1351 | m/z(ES-) = 1988 | m/z(ES-) = 1433 | m/z(ES-) = 1359 | m/z(ES-) = 1504 (z = 3) |
| | | | (z = 2) | (z = 2) | |
| H | C₄₄H₆₆N₁₀O₁₄Tm₂ | C₆₃H₉₆N₁₅O₂₁Tm₃ | C₁₀₁H₁₄₀N₂₀O₂₈Tm₄ | C₈₆H₁₄₄N₂₂O₂₈Tm₄ | C₁₅₀H₂₁₀N₃₃O₄₈P₃Tm₆ |
| | m/z(ES-) = 1295 | m/z(ES-) = 1904 | m/z(ES-) = 1377 | m/z(ES-) = 1303 | m/z(ES-) = 1448 (z = 3) |
| | | | (z = 2) | (z = 2) | |
| I | C₆₈H₇₂N₁₆O₂₂Tm₂ | C₉₉H₁₀₅N₂₄O₃₃Tm₃ | C₁₄₉H₁₅₂N₃₂O₄₄Tm₄ | C₁₃₄H₁₅₆N₃₄O₄₄Tm₄ | C₂₂₂H₂₂₈N₅₁O₇₂P₃Tm₆ |
| | m/z(ES-) = 1801 | m/z(ES-) = 2663 | m/z(ES-) = 1883 | m/z(ES-) = 1809 | m/z(ES-) = 1954 (z = 3) |
| | | | (z = 2) | (z = 2) | |
| J | C₃₈H₄₈N₈O₁₈Tm₂ | C₅₄H₆₉N₁₂O₂₇Tm₃ | C₈₉H₁₀₄N₁₆O₃₆Tm₄ | C₇₄H₁₀₈N₁₈O₃₆Tm₄ | C₁₃₂H₁₅₆N₂₇O₆₀P₃Tm₆ |
| | m/z(ES-) = 1241 | m/z(ES-) = 1823 | m/z(ES-) = 1323 | m/z(ES-) = 1249 | m/z(ES-) = 1394 (z = 3) |
| | | | (z = 2) | (z = 2) | |

### Example 25

### Condensation of Aromatic Amines (R1-NH) on Polyacid Cores R′(COOH)ₙ

R′(COOH)ₙ+nR1-NH₂→R′(CONH—R1)ₙ

1 mmol of the acid compound and 1.1 equivalents per acid function of the amine compound (R3-NH₂) are dissolved in a 25/75 v/v mixture of H₂O/DMSO. After dissolution, 384 mg (2 mmol per acid function) of EDCI and 79 mg (0 6 mmol) of HOBT are added. The reaction medium is stirred for 18 h at ambient temperature, the pH being maintained at 6. The reaction medium is poured dropwise into ethanol. The precipitate obtained is filtered off and washed with ether.

The product is purified by ultrafiltration through a 1 KD membrane or by preparative HPLC.

Compounds obtained with R1-NH₂=compound A, B, C or D:

| | I | II | III | IV | V |
|---|---|---|---|---|---|
| A | C₅₈H₆₂N₁₀O₁₄Tm₂ | C₈₄H₉₀N₁₅O₂₁Tm₃ | C₁₂₉H₁₃₂N₂₀O₂₈Tm₄ | C₁₁₄H₁₃₆N₂₂O₂₈Tm₄ | C₁₉₂H₁₉₈N₃₃O₄₈P₃Tm₆ |
| | m/z(ES-) = 1459 | m/z(ES-) = 2150 | m/z(ES-) = 1541 | m/z(ES-) = 1467 | m/z(ES-) = 1612 (z = 3) |
| | | | (z = 2) | (z = 2) | |
| B | C₅₈H₆₂N₁₀O₁₄Tm₂ | C₈₄H₉₀N₁₅O₂₁Tm₃ | C₁₂₉H₁₃₂N₂₀O₂₈Tm₄ | C₁₁₄H₁₃₆N₂₂O₂₈Tm₄ | C₁₉₂H₁₉₈N₃₃O₄₈P₃Tm₆ |
| | m/z(ES-) = 1459 | m/z(ES-) = 2150 | m/z(ES-) = 1541 | m/z(ES-) = 1467 | m/z(ES-) = 1612 (z = 3) |
| | | | (z = 2) | (z = 2) | |
| C | C₅₆H₇₄N₁₀O₁₄Tm₂ | C₈₁H₁₀₈N₁₅O₂₁Tm₃ | C₁₂₅H₁₅₆N₂₀O₂₈Tm₄ | C₁₁₀H₁₆₀N₂₂O₂₈Tm₄ | C₁₈₆H₂₃₄N₃₃O₄₈P₃Tm₆ |
| | m/z(ES-) = 1447 | m/z(ES-) = 2132 | m/z(ES-) = 1529 | m/z(ES-) = 1455 | m/z(ES-) = 1600 (z = 3) |
| | | | (z = 2) | (z = 2) | |
| D | C₅₀H₆₂N₁₀O₁₄Tm₂ | C₇₂H₉₀N₁₅O₂₁Tm₃ | C₁₁₃H₁₃₂N₂₀O₂₈Tm₄ | C₉₈H₁₃₆N₂₂O₂₈Tm₄ | C₁₆₈H₁₉₈N₃₃O₄₈P₃Tm₆ |
| | m/z(ES-) = 1363 | m/z(ES-) = 2006 | m/z(ES-) = 1445 | m/z(ES-) = 1371 | m/z(ES-) = 1516 (z = 3) |
| | | | (z = 2) | (z = 2) | |

### Example 26

### Condensation of Carboxylic Acids on Polyamine Cores R″(NH₂)ₙ

R″(NH₂)ₙ+nR3-COOH→R″(NHCO—R3)ₙ

Compounds obtained with R3-COOH=compound K, K′, L, M, N, O, P or Q:

| | VI | VII | VIII | IX | X |
|---|---|---|---|---|---|
| K | C₄₂H₅₄N₁₀O₁₄Tm₂ | C₆₆H₈₇N₁₆O₂₁Tm₃ | C₇₅H₁₀₂N₁₉O₂₄Tm₃ | C₉₆H₁₃₂N₂₂O₂₈Tm₄ | C₁₀₂H₁₄₀N₂₆O₃₂Tm₄ |
| | m/z(ES-) = 1259 | m/z(ES-) = 972 | m/z(ES-) = 1079 | m/z(ES-) = 1357 | m/z(ES-) = 1457 |
| | | (z = 2) | (z = 2) | (z = 2) | (z = 2) |
| K′ | C₄₂H₅₄N₁₀O₁₄Dy₂ | C₆₆H₈₇N₁₆O₂₁Dy₃ | C₇₅H₁₀₂N₁₉O₂₄Dy₃ | C₉₆H₁₃₂N₂₂O₂₈Dy₄ | C₁₀₂H₁₄₀N₂₆O₃₂Dy₄ |
| | m/z(ES-) = 1246 | m/z(ES-) = 962 | m/z(ES-) = 1069 | m/z(ES-) = 1344 | m/z(ES-) = 1444 |
| | | (z = 2) | (z = 2) | (z = 2) | (z = 2) |
| L | C₄₄H₅₈N₁₀O₁₄Tm₂ | C₆₉H₉₃N₁₆O₂₁Tm₃ | C₇₈H₁₀₈N₁₉O₂₄Tm₃ | C₁₀₀H₁₄₀N₂₂O₂₈Tm₄ | C₁₀₆H₁₄₈N₂₆O₃₂Tm₄ |
| | m/z(ES-) = 1287 | m/z(ES-) = 993 | m/z(ES-) = 1100 | m/z(ES-) = 1385 | m/z(ES-) = 1485 |
| | | (z = 2) | (z = 2) | (z = 2) | (z = 2) |
| M | C₄₀H₅₀N₁₀O₁₄Tm₂ | C₆₃H₈₁N₁₆O₂₁Tm₃ | C₇₂H₉₆N₁₉O₂₄Tm₃ | C₉₂H₁₂₄N₂₂O₂₈Tm₄ | C₉₈H₁₃₂N₂₆O₃₂Tm₄ |
| | m/z(ES-) = 1231 | m/z(ES-) = 951 | m/z(ES-) = 1058 | m/z(ES-) = 1329 | m/z(ES-) = 1429 |
| | | (z = 2) | (z = 2) | (z = 2) | (z = 2) |
| N | C₄₀H₅₀N₁₀O₁₆Tm₂ | C₆₃H₈₁N₁₆O₂₄Tm₃ | C₇₂H₉₆N₁₉O₂₇Tm₃ | C₉₂H₁₂₄N₂₂O₃₂Tm₄ | C₉₈H₁₃₂N₂₆O₃₆Tm₄ |
| | m/z(ES-) = 1263 | m/z(ES-) = 975 | m/z(ES-) = 1082 | m/z(ES-) = 1361 | m/z(ES-) = 1461 |
| | | (z = 2) | (z = 2) | (z = 2) | (z = 2) |
| O | C₅₈H₆₂N₁₄O₂₀Tm₂ | C₉₀H₉₉N₂₂O₃₀Tm₃ | C₉₉H₁₁₄N₂₅O₃₃Tm₃ | C₁₂₈H₁₄₈N₃₀O₄₀Tm₄ | C₁₃₄H₁₅₆N₃₄O₄₄Tm₄ |
| | m/z(ES-) = 1611 | m/z(ES-) = 1236 | m/z(ES-) = 1343 | m/z(ES-) = 1709 | m/z(ES-) = 1809 |
| | | (z = 2) | (z = 2) | (z = 2) | (z = 2) |
| P | C₆₄H₇₂N₁₆O₂₂Tm₂ | C₉₉H₁₁₄N₂₅O₃₃Tm₃ | C₁₀₈H₁₂₉N₂₈O₃₆Tm₃ | C₁₄₀H₁₆₈N₃₄O₄₄Tm₄ | C₁₄₆H₁₇₆N₃₈O₄₈Tm₄ |
| | m/z(ES-) = 1753 | m/z(ES-) = 1343 | m/z(ES-) = 1449 | m/z(ES-) = 1851 | m/z(ES-) = 1951 |
| | | (z = 2) | (z = 2) | (z = 2) | (z = 2) |
| Q | C₃₄H₄₈N₈O₁₈Tm₂ | C₅₄H₇₈N₁₃O₂₇Tm₃ | C₆₃H₉₃N₁₆O₃₀Tm₃ | C₈₀H₁₂₀N₁₈O₃₆Tm₄ | C₈₆H₁₂₈N₂₂O₄₀Tm₄ |
| | m/z(ES-) = 1193 | m/z(ES-) = 923 | m/z(ES-) = 1029 | m/z(ES-) = 1291 | m/z(ES-) = 1391 |
| | | (z = 2) | (z = 2) | (z = 2) | (z = 2) |

### Example 27

### Condensation of Isothiocyanates on Polyamine Cores R″(NH₂)ₙ

R″(NH₂)ₙ+nR4-NCS→R″(NH—(C═S)—NHR4)ₙ

The isothiocyanate compound (1.5 mmol) is dissolved at ambient temperature in 20 ml of DMSO. The polyamine core (n=2: 0.68 mmol; n=3: 0.45 mmol; n=4: 0.34 mmol) is then added and the reaction medium is stirred for 48 h, before being precipitated from 200 ml of ethyl ether. The precipitate is washed with ethyl ether and then ethanol. The product is then purified on silica.

Compounds obtained with R4-NCS=compound R or S:

| | VI | VII | VIII | IX | X |
|---|---|---|---|---|---|
| R | C₄₆H₆₄N₁₂O₁₂S₂Tm₂ | C₇₂H₁₀₂N₁₉O₁₈S₃Tm₃ | C₈₁H₁₁₇N₂₂O₂₁S₃Tm₃ | C₁₀₄H₁₅₂N₂₆O₂₄S₄Tm₄ | C₁₁₀H₁₆₀N₃₀O₂₈S₄Tm₄ |
| | m/z(ES-) =1377 | m/z(ES-) =1061 | m/z(ES-) = 1167 | m/z(ES-) = 1475 | m/z(ES-) = 1575 |
| | | (z = 2) | (z = 2) | (z = 2) | (z = 2) |
| S | C₅₄H₆₄N₁₂O₁₂S₂Tm₂ | C₈₄H₁₀₂N₁₉O₁₈S₃Tm₃ | C₉₃H₁₁₇N₂₂O₂₁S₃Tm₃ | C₁₂₀H₁₅₂N₂₆O₂₄S₄Tm₄ | C₁₂₆H₁₆₀N₃₀O₂₈S₄Tm₄ |
| | m/z(ES-) =1473 | m/z(ES-) = 1133 | m/z(ES-) = 1167 | m/z(ES-) = 1571 | m/z(ES-) = 1671 |
| | | (z = 2) | (z = 2) | (z = 2) | (z = 2) |

### Example 28

### Condensation of Squarates on Polyamine Cores R″(NH₂)ₙ

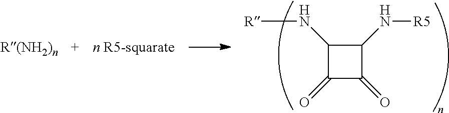

The squarate compound (1.5 mmol) is dissolved at ambient temperature in 20 ml of DMSO. The polyamine core (n=2: 0.68 mmol; n=3: 0.45 mmol; n=4: 0.34 mmol) and also 1 5 mmol of triethylamine are then added, and the reaction mixture is stirred for 24 h at 50° C., before being precipitated from 200 ml of ethyl ether. The precipitate is washed with ethyl ether and then ethanol. The product is then purified on silica. Compounds obtained with R5-squarate=compound T, U or V:

| | VI | VII | VIII | IX | X |
|---|---|---|---|---|---|
| T | C₅₀H₆₀N₁₂O₁₆Tm₂ | C₇₈H₉₆N₁₉O₂₄Tm₃ | C₈₇H₁₁₁N₂₂O₂₇Tm₃ | C₁₁₂H₁₄₄N₂₆O₃₂Tm₄ | C₁₁₈H₁₅₂N₃₀O₃₆Tm₄ |
| | m/z(ES-) = 1421 | m/z(ES-) = 1094 | m/z(ES-) = 1200 | m/z(ES-) = 1519 | m/z(ES-) = 1619 |
| | | (z = 2) | (z = 2) | (z = 2) | (z = 2) |
| U | C₅₀H₆₀N₁₂O₁₆Tm₂ | C₇₈H₉₆N₁₉O₂₄Tm₃ | C₈₇H₁₁₁N₂₂O₂₇Tm₃ | C₁₁₂H₁₄₄N₂₆O₃₂Tm₄ | C₁₁₈H₁₅₂N₃₀O₃₆Tm₄ |
| | m/z(ES-) = 1421 | m/z(ES-) = 1094 | m/z(ES-) = 1200 | m/z(ES-) = 1519 | m/z(ES-) = 1619 |
| | | (z = 2) | (z = 2) | (z = 2) | (z = 2) |
| V | C₅₀H₇₆N₁₂O₁₆Tm₂ | C₇₈H₁₂₀N₁₉O₂₄Tm₃ | C₈₇H₁₃₅N₂₂O₂₇Tm₃ | C₁₁₂H₁₇₆N₂₆O₃₂Tm₄ | C₁₁₈H₁₈₄N₃₀O₃₆Tm₄ |
| | m/z(ES-) = 1437 | m/z(ES-) = 1106 | m/z(ES-) = 1212 | m/z(ES-) = 1535 | m/z(ES-) = 1635 |
| | | (z = 2) | (z = 2) | (z = 2) | (z = 2) |

### PART IV/SYNTHESIS OF LIPOPHILIC COMPLEXES FOR INCORPORATION IN AN ENCAPSULATING SYSTEM (EMULSION OR LIPOSOME, IN PARTICULAR)

### Example 29

### Chelate q=2 Complex with Phosholipid Membrane Anchoring Group

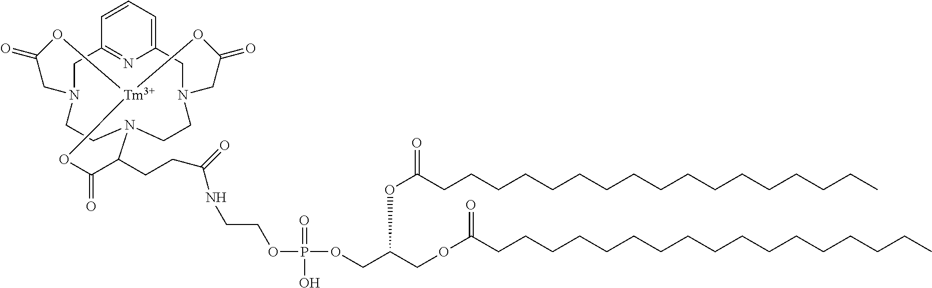

The synthesis is identical to that described in example 5 of patent WO 2006/100305, the complexation being carried out (as stage i, ex1) with TmCl₃. 6H₂O.

C₆₁H₁₀₅N₅O₁₅PTm

Maldi-T of (negative mode): m/z=1346.

### Example 30

### q=2

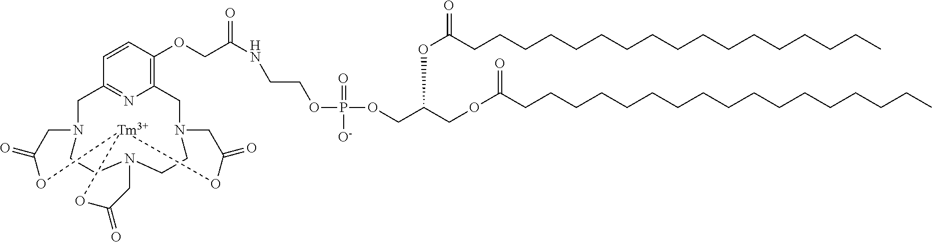

The synthesis is identical to that described in example 8 of patent WO 2006/100305, the complexation being carried out with TmCl₃. 6H₂O.

C₅₆H₉₄N₅O₁₆PTm

Maldi-T of (negative mode): m/z=1291.

### Example 31

### q=2

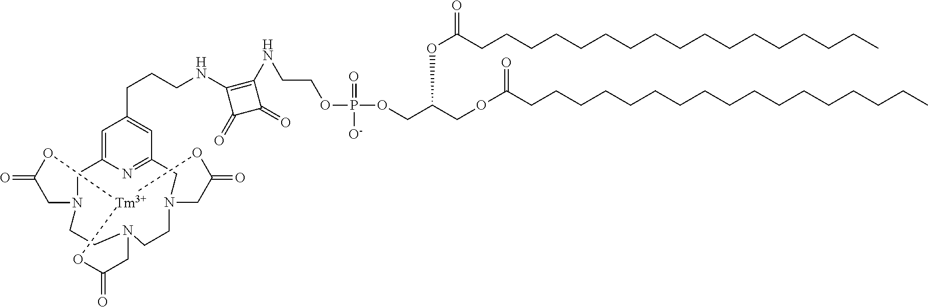

The synthesis is identical to that described in example 19 of patent WO 2006/100305, the complexation being carried out with TmCl₃. 6H₂O.

C₆₅H₁₀₈N₅O₁₆PTm

Maldi-T of (negative mode): m/z=1427.

### Example 32

### q=2

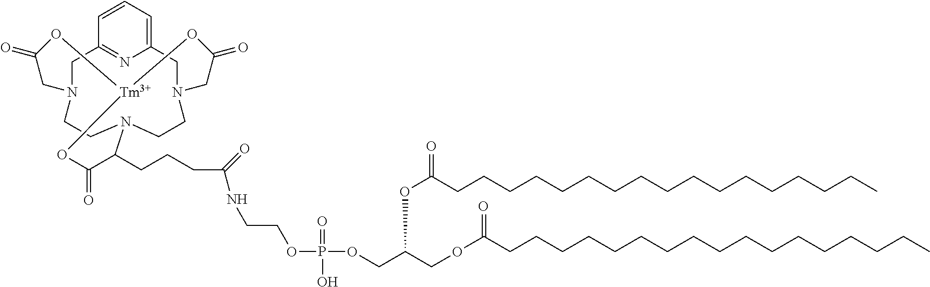

The synthesis is identical to that described in example 21 of patent WO 2006/100305, the complexation being carried out with TmCl₃. 6H₂O.

C₅₈H₉₉N₅O₁₅PTm

Maldi-T of (negative mode): m/z=1304.

### Example 33

The compounds obtained in this example 33 are in particular of use as membrane chelates used for nanoemulsions thanks to the particular lipophilic chains used. The example is detailed for DOTA chelate, similar adapted protocol is used for q=2 chelates.

### a) Synthesis of (2-{4-dioctadecylcarbamoylmethyl-7,10-bis[(ethoxycarbonylmethylcarbamoyl)methyl]-1,4,7,10-tetraazacyclododec-1-yl}acetylamino)acetic acid ester

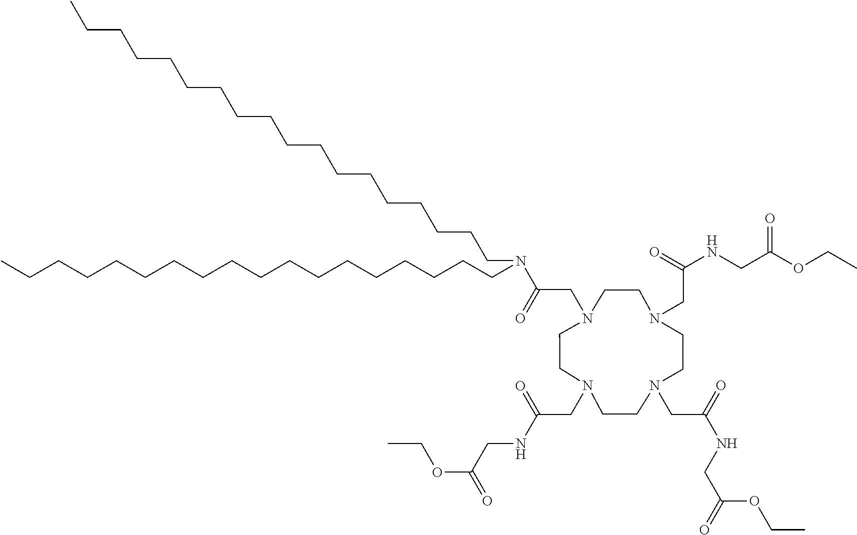

In a 25 mL round-bottom flask, 80 mg of the intermediate

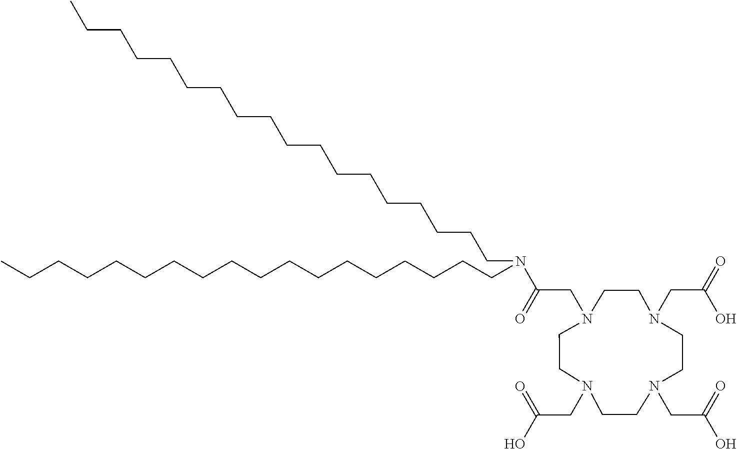

(0.09 mmol; 1 equiv) and 27 mg of glycine ethyl ester (0.26 mmol; 3 equiv) are dissolved in 4 mL of chloroform CHCl₃. 121 mg of HBTU (0.32 mmol; 3.5 equiv) and 97 mg of DMAP (0.79 mmol; 8.8 equiv). The reaction medium is then at 30° C. for 2 days and is then evaporated to dryness. The product is washed with water and then filtered (88.8 mg).

C₆₄H₁₂₂N₈O₁₀; MALDI-TOF: Positive mode: m/z 1163.94.

### b) Synthesis of (2-{4,7-bis[(carboxymethylcarbamoyl)methyl]-10-dioctadecylcarbamoylmethyl-1,4,7,10-tetraazacyclododec-1-yl}acetylamino)acetic acid

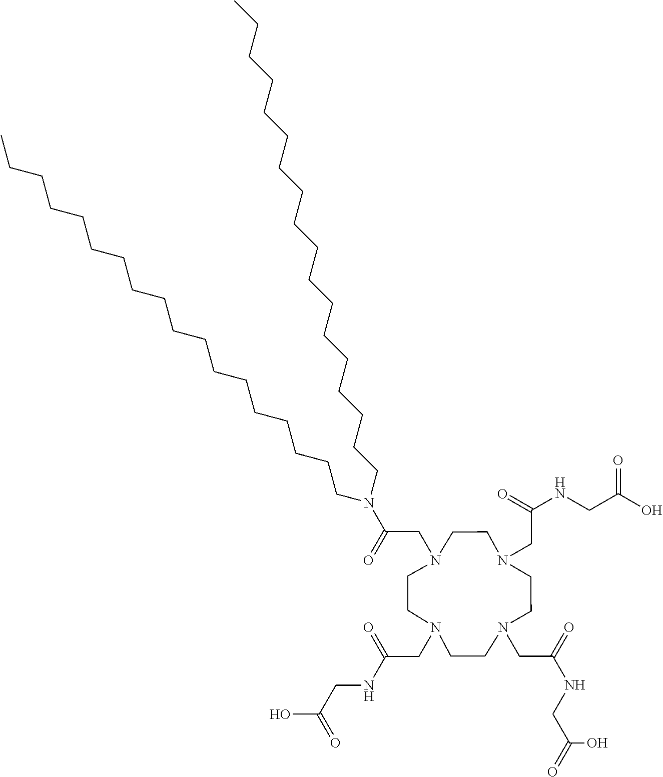

40 mg of the intermediate obtained in f) (0.03 mmol; 1 equiv) are dissolved in 20 mL of a 1/1 (v/v) mixture: 1 concentrated HCl/dioxane. The reaction medium is then stirred for 2 hours at ambient temperature.

After evaporation of the solvent to dryness and washing with water, 27.7 mg of a white powder are obtained.

C₅₈H₁₁₀N₈O₁₀; MALDI-TOF: Positive mode m/z 1079.83.

### c) Synthesis of Lanthanide Complexes

### Example with Lipophilic Aliphatic Chain

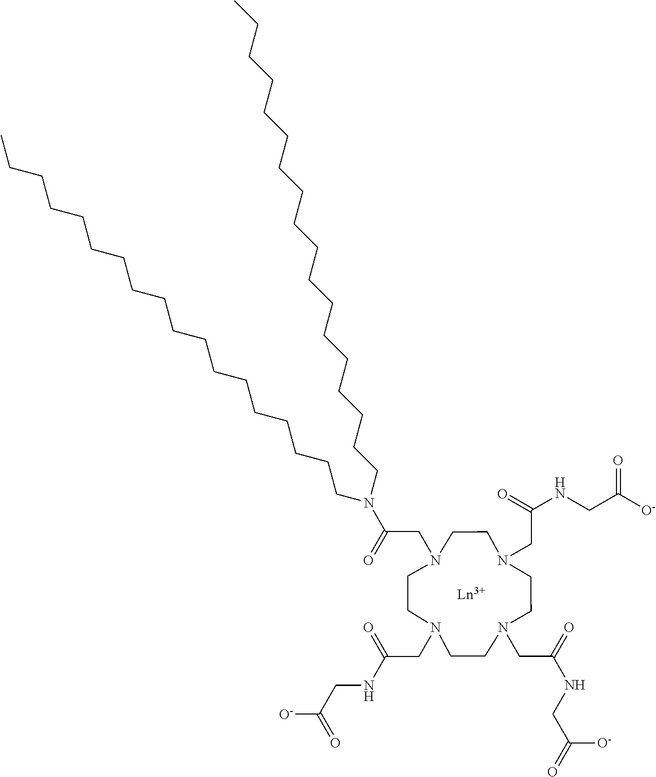

100 mg of the intermediate obtained in a) (0.09 mmol; 1 equiv) are dissolved in 2 mL of CH₃OH. 0.1 mmol (1.1 eq) of lanthanide chloride (EuCl₃. 6H₂O, TmCl₃. 6H₂O, YbCl₃. 6H₂O) is then added. The pH is adjusted to 7 by adding 0.1 N sodium acetate in MeOH. The reaction medium is refluxed for 1 h. After evaporation to dryness, the product is washed with water and then filtered. 100 mg of a white powder are obtained.

| | | m/z (Maldi-Tof) |
|---|---|---|
| Ln³⁺ | Formula | negative |
| Eu³⁺ | C₅₈H₁₀₇N₈O₁₀Eu | 1226 |
| Tm³⁺ | C₅₈H₁₀₇N₈O₁₀Tm | 1244 |
| Yb³⁺ | C₅₈H₁₀₇N₈O₁₀Yb | 1247 |
| Dy³⁺ | C₅₈H₁₀₇N₈O₁₀Dy | 1237 |
| Gd³⁺ | C₅₈H₁₀₇N₈O₁₀Gd | 1231 |

The product obtained carries amide functions active for Cest imaging. The chelate is situated at the external face of the emulsion droplet nanoparticle.

### PART V) EXAMPLES 34 TO 37

### Preparation of Targeted Liposomes that Comprise Targeting Entities (Biovectors)

These examples 34 to 36 illustrate the preparation of peptides coupled with a lipophilic anchoring group that allows the peptide to be attached on the surface of the liposome (for specific targeting). Several non limitative examples of linkers are shown (squarate, PEG-squarate, glycine amino acid; PEG groups or alkylene group in particular (CH2)_{1 to 5}, notably (CH2)₂ are used with the same adapted protocol). The lipophilic group (phospholipid, cholesterol) is inserted into the membrane of the liposome as described below.

### Example 34

### a) Step 1

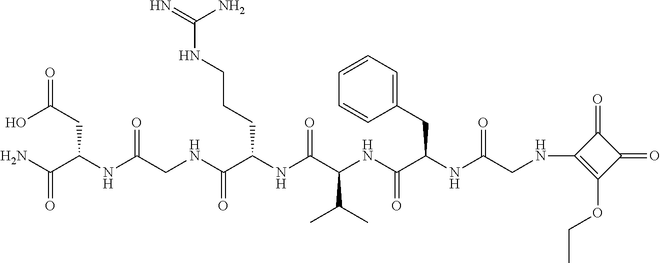

100 mg (0.15 mmol) of peptide H-Gly-(D)-Phe-(L)-Val-(L)-Arg-Gly-(L)Asp-NH₂ (H-GfVRGD-NH₂) bought from Bachem were dissolved in 3 ml of DMSO under argon. 23 μl of 3,4-Diethoxy-3-cyclobutene-1,2-dione (0.15 mmol; 1 eq) and 25 μl of triethylamine were added. The reaction were mixed overnight at 40° C. before being precipitated in 40 ml of diethyl ether. After filtration, 98 mg of powder are obtained (Yield: 84%).

C₃₄H₄₈N₁₀O₁₁; m/z=773 (ES+)

### b) Step 2

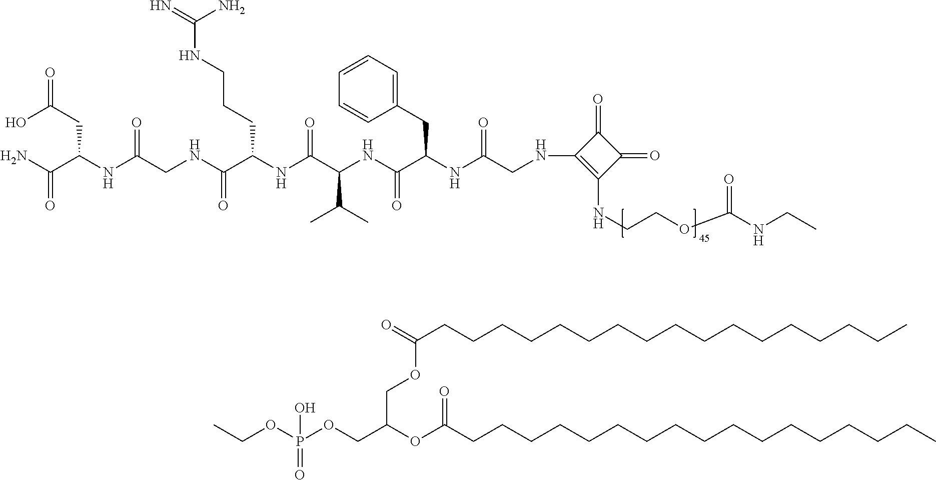

95 mg of product obtained at the step a) (0.12 mmol; 1 equiv) and 430 mg (0.15 mmol, 1.25 eq) of 1,2-distearoyl-sn-glycero-3-phosphoethanolamine-N-[amino(polyethyleneglycol)-2000] (ammonium salt) were dissolved in 3 mL of DMSO with 25 μl of triethylamine. The reaction were mixed during 48h at room temperature. The product is precipitated in 40 ml of diethyl ether. After filtration, we obtained 400 mg of powder. The product is purified on a C4 column on a flash chromatography system with a gradient of ammonium formiate 10 mM pH6/Methanol. 260 mg of white powder are obtained (yield: 62%).

C₁₆₄H₃₀₅N₁₂O₆₄P; MALDI-TOF positif Mode m/z=3501

### Example 35

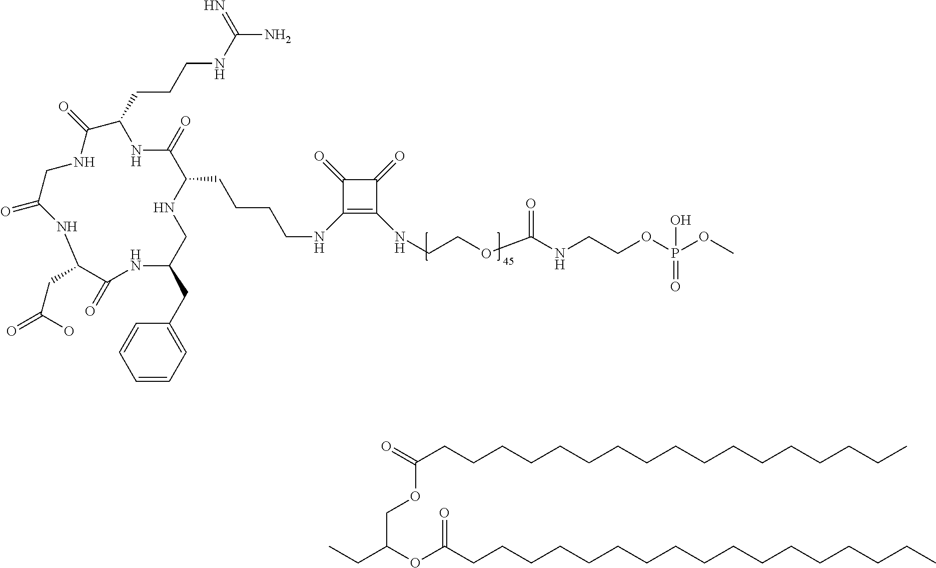

The same protocole as described in the example 34 is used, with 90 mg of the cyclic peptide RGDfK bought from Bachem.

C₁₆₃H₃₀₂N₁₁O₆₃P; MALDI-TOF: positif mode m/z=3456

Examples 34 and 35 are prepared in a similar way with other linkers (no squarate or PEG), for instance alkylene linker.

### Example 36

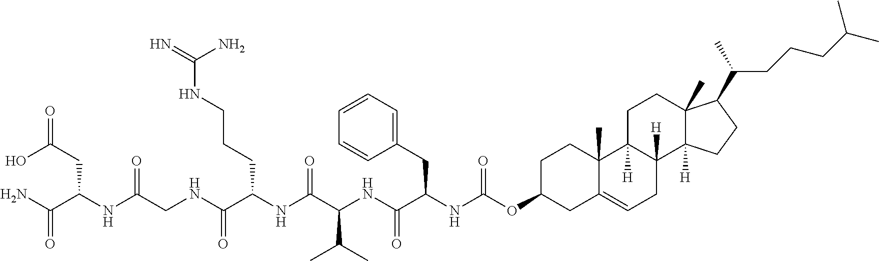

100 mg (0.15 mmol) of peptide H-Gly-(D)-Phe-(L)-Val-(L)-Arg-Gly-(L)Asp-NH₂ (H-GfVRGD-NH₂) synthesized by Bachem, 63 mg (0.14 mmol) of cholesteryl chloroformate and 25 μl of triethylamine were dissolved in 5 ml of dichloromethane. The reaction is mixed during 48 h at room temperature. The product is precipitated in 40 ml of diethyl ether. After filtration, the precipitate is washed with acidic water. The product thus obtained is then purified on a C4 column on a flash chromatography system with a gradient of ammonium formiate 10 mM pH6/Methanol. 100 mg of white powder are obtained (yield: 63%).

The Gly is the linker used.

C₅₆H₈₈N₁₀O₁₀; MALDI-TOF: positif mode m/z=1061

### Example 37

### Preparation of Spherical Liposomes with Biovector on the Surface

A lipid solution containing 55 mol % POPC, 5% DPPG, 34% cholesterol, 5% DSPE-PEG₂₀₀₀ and 1% of the compound of the Example 34 was prepared in a chloroform-methanol mixture at room temperature. The solution is then evaporated and the lipid film thus obtained is dried under vacuum overnight before being rehydrated at 55° C. with an aqueous 300 mM of complex (monomeric complex q=2 or multimer of monomeric complex q=2) solution previously filtered on 0.22 μm. The liposomal solution obtained is then extruded at 45° C. successively on filter 1 μm, 0.8 μm, 0.4 μm and 0.2 μm. Finally the liposomes were purified by size exclusion chromatography on Sephadex G25M cartridges (GE).

The same experimental protocol is used with varying proportions of the compound of the Example 34, 35 or 36 from 1 to 15 mol %.

### PART VI) SYNTHESIS OF LIPOSOMES INCORPORATING CHELATES

### Spherical Liposomes

2.5 mL of a solution of lipid at a total concentration of 25 mg/ml, in a chloroform-methanol mixture, are prepared in the following proportions: 55% POPC (34 mg), 5% DPPG (3 mg) and 40% cholesterol (25 mg).

The solution is then evaporated to dryness and the lipid film thus obtained is dried under vacuum overnight, before being rehydrated with 2.5 ml of an aqueous solution, at pH7, of complex (monomeric complex q=2 or multimer of monomeric complex q=2) at 300 mOsm/kg, prefiltered through 0.22 μm. The solution is stirred for 1 h at 40° C. with ceramic beads. It is then subjected to ultrasound for 10 min. This solution is then extruded successively through filters of 1 μm, 0.8 μm and 0.4 μm and, finally, 0.2 μm, with heating to 45° C. Finally, the liposomes are purified on a size exclusion gel on Sephadex G25M cartridges (GE).

| | Hydrodynamic | | |
|---|---|---|---|
| | diameter | Osmolality | [Tm] |
| Complex | (nm) | (mOsm/kg) | mM |
| DO3A-Tm | 154 | 222 | 9.8 |
| 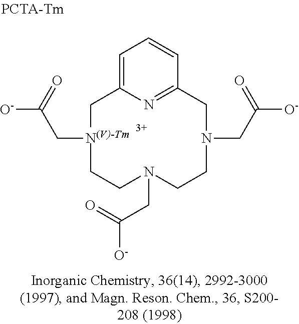 | 195 | 308 | 6.1 |
| DOTA-Tm | 172 | 272 | 2.5 |
| DOTMA-Tm | 179 | | 2.9 |

The same experimental protocol is used for instance with the following surfactant compositions:

### Egg PC/Cholesterol/DSPE-PEG2000 75/20/5

### Egg PC/Cholesterol 80/20

### Egg PC/DSPE-PEG2000 95/5

### DMPC/Cholesterol/DSPE-PEG2000 75/20/5

### DMPC/DSPE-PEG2000 95/5

### DMPC/cholesterol 80/20.

### DPPC/POPC/DSPE-PEG2000: 90/5/5; 85/10/5; 80/15/5; 75/20/5

### Non-Spherical Liposomes

A solution of lipid containing 75 mol % DPPC, 20 mol % of lipophilic complex and 5 mol % of DSPE-PEG2000 is dissolved in chloroform at ambient temperature.

The solution is then evaporated to dryness and the lipid film thus obtained is dried under vacuum overnight, before being rehydrated at 55° C. with a 40 mM aqueous solution of q=2 (monomeric chelate or multimer of monomeric chelate) complex, prefiltered through 0.22 μm. This solution is then extruded six times through 0.2 μm filters, with heating at 55° C. The liposomal suspension is then dialysed against an isotonic solution (3000sm, pH=7.4) in order to purify the liposome and to render it asymmetrical.

### CEST Emulsion (Nanoemulsion-Nanodroplets)

The nanoparticles are obtained by emulsifying 10 to 20% (v/v) for instance 10 or 15% of perfluorooctylbromide (PFOB), 1 to 10% (w/v) for instance 5% of a mixture of surfactant (phosphatidylcholine/dipalmitoylphosphatidylethanolamine and the lipophilic complex q=2, for example in a 59/1/40 ratio) and 2.5% (w/v) of glycerol and water. This mixture is emulsified preferably for 4 minutes at 20000 psi.

### PART VII) CEST IMAGING PROPERTIES OF LIPOSOMES ENCAPSULATING q=2 MONOMERIC CHELATES, IN PARTICULAR OF SPHERICAL LIPOSOMES AS PRODUCED ABOVE

### 1) PCTA-Tm Chelates

Z-spectrum spectra at 310 K and 300 MHz

Experimental conditions:

SW 75 ppm, TD:32768, NS:1 or 4, DS:2, AQ:0.72 s, D1=15 s saturation for 3 s.

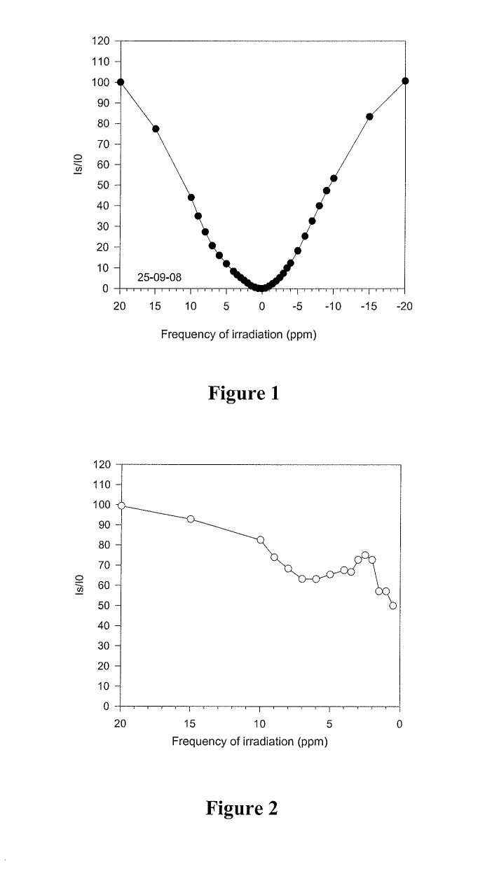

FIG. 2 demonstrates a peak around 8 ppm, which demonstrates the effectiveness of the product (liposome containing free PCTA-Tm q=2 chelate). The intensity of the water peak is measured at an irradiation power of 31 dB (FIGS. 1 and 2).

### 2) DO3A-Tm Chelates

Z-spectrum spectra at 310 K and 300 MHz

Experimental conditions:

SW 75 ppm, TD:32768, NS:1 or 4, DS:2, AQ:0.72 s, D1=15 s saturation for 3 s.

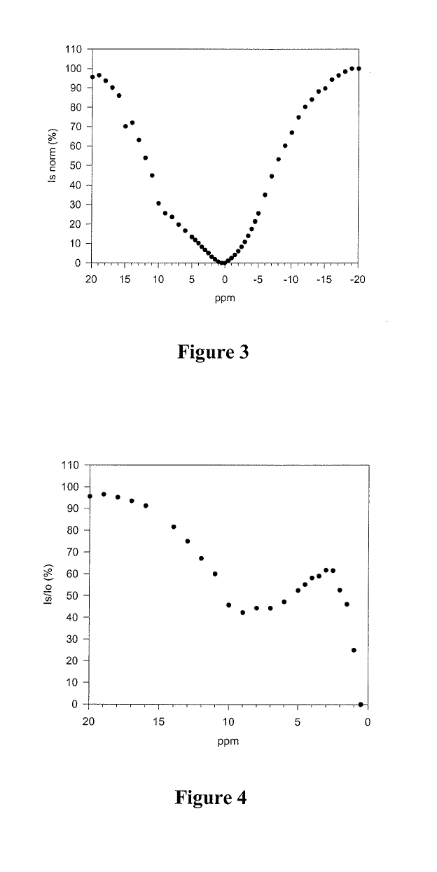

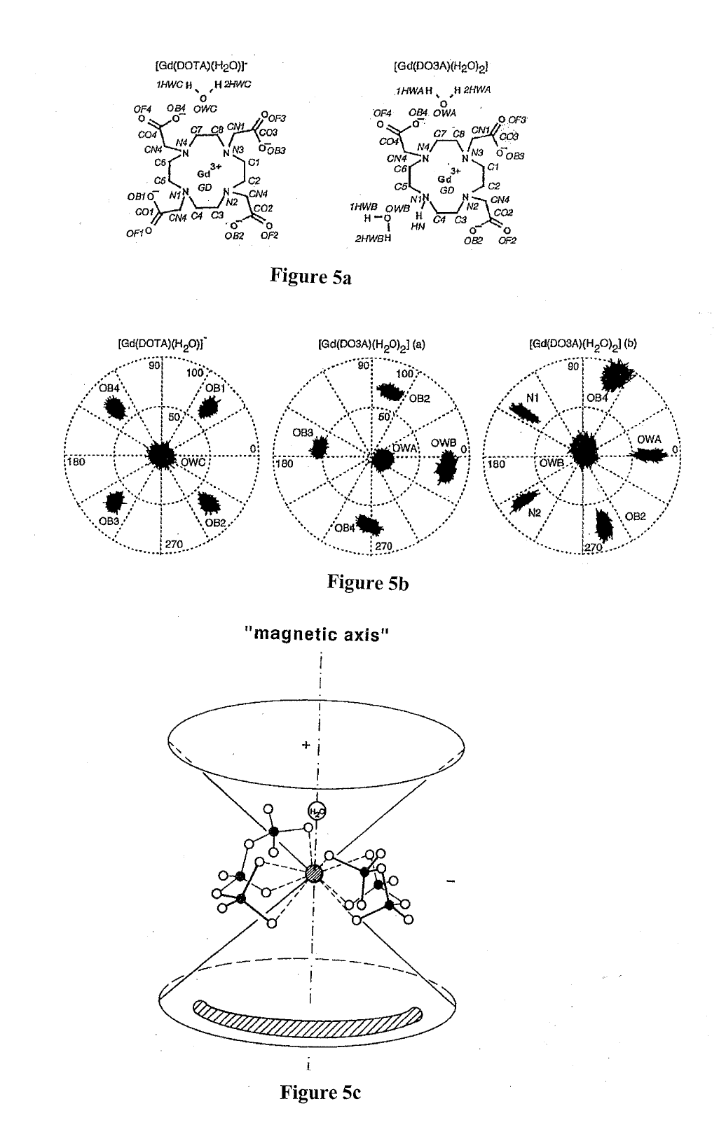

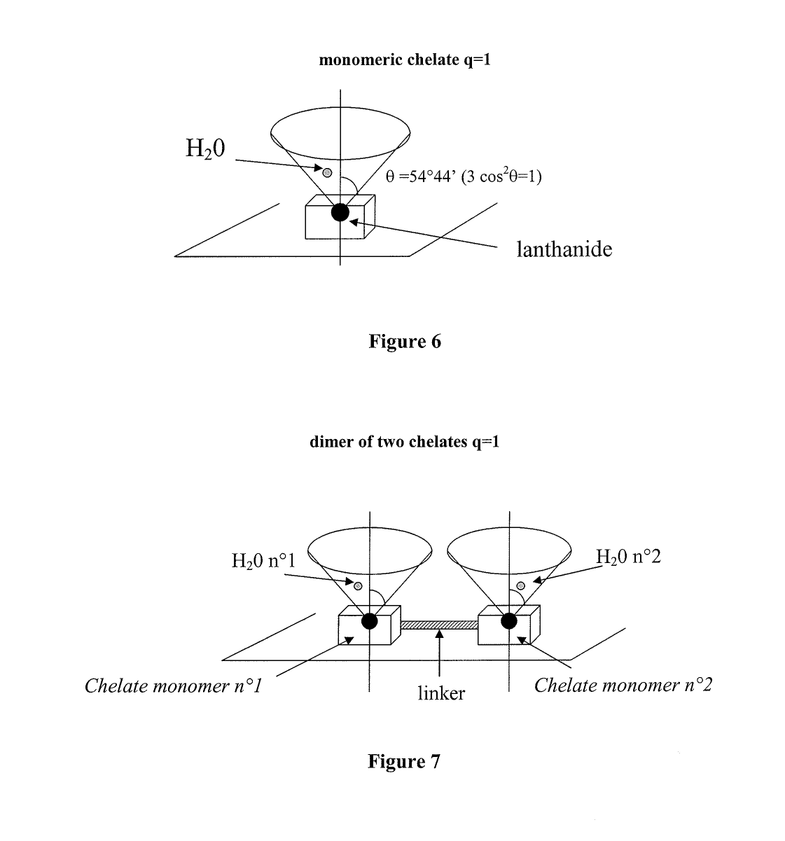

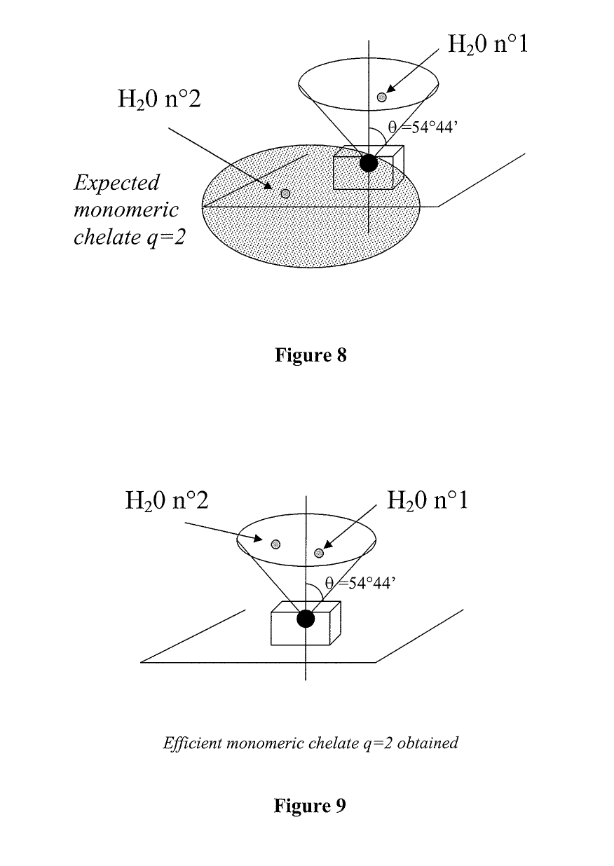

FIG. 4 demonstrates a peak around 8 ppm, which demonstrates the effectiveness of the product.

The intensity of the water peak is measured at an irradiation power of 31 dB (FIGS. 3 and 4).

In comparison, the prior art compounds DOTA-Tm and DOTMA-Tm (of q=1 type) give a delta (peak) shift value of respectively, 4 and 1.8, i.e. much lower than the values for the chelate compositions according to the present invention (peak at 8 ppm).

### PART VIII) CEST IMAGING WITH COMPOUNDS OF THE INVENTION

### In Vivo Imaging

1) In Vivo Images—Rodents Brain—CEST Liposomes (Chelate DO3A q=2)

Phantom and Animal preparation: In vitro experiments were performed on brain homogenates embedded in 4% agarose matrix with various macromolecules (0.8/1.6/3.2/6.5% wt) and liposomes of the applicant containing DO3A chelate. The shift obtained was 9 ppm. The concentrations of liposomes were C_{CEST liposomes}=0/5/10/25 nM. CEST liposomes intracerebral injections (V=3 μL, C_{CEST liposomes}=50 nM, shift=12 ppm) were performed in two anesthetized rats.

MRI acquisition: In vivo Z-spectra and CEST images were acquired on a 7 T Pharmascan MRI scanner using a volume coil with a CEST efficient sequence (TE/TR=54/5000 ms, T_{acq}=14 min) preceded by a Continuous Wave saturation pulse (Tₛₐₜ=400 ms, B₁ₛₐₜ˜7 μT) being applied at ±12 ppm in vivo and ±9 ppm in vitro.

Image analysis: Liposomes CEST concentration maps were calculated using an image analysis tool programmed with Matlab which simulates the overall (endogenous MT+exogenous CEST) asymmetric Z-spectra.

### Results

Liposomes CEST concentrations used lead to images acquired in the rodent brain in vivo.

### 2) Example—In Vivo Images—Mouse Brain—CEST Integrin Targeted Liposomes

(chelate DO3A q=2; biovector is RGD peptide targeting integrin over-expressed in tumor)

### Subjects and Methods

Animal preparation. Tumor was induced by i.c. injection of 1.2×10⁵ Glioma U87 human cells in a single immuno-depressed “nude” mouse brain [Moats R A et al., Mol Imaging, 2, 150-8.]

Experiments were performed 10 days after.

MRI acquisition. Brain CEST images were acquired using a CEST appropriated sequence (TE/TR=54/5000 ms, resolution 150×150×660 μm³, Tacq=14 min) preceded by a Continuous Wave saturation pulse (Tₛₐₜ=400 ms, B₁ₛₐₜ˜7 μT, δₛₐₜ=±9 ppm) on a 7 T small animal MRI scanner (Bruker, Germany) using an home-made 2.8 cm-diameter quadrature volumic ¹H coil. Images were acquired before (pre-injection) and 1-hr (post-injection) after i.v injection of 200 μL of RGD-CEST-liposomes (of example 37) in the tail vein.

Image analysis. % CEST images were obtained by the subtraction of images acquired with saturation applied at 9 and −9 ppm normalized by the reference image without saturation. % CEST contrast was analyzed in different regions-of-interest corresponding to: the entire “brain”, the “tumor and its surroundings” and the area “controlateral” to the tumor.

### Results

The average % CEST contrast before injection in the “tumor” is 3.9% (corresponding to the endogenous MT background effect) and rise to 7.2% after injection which corresponds to an 84% elevation of the % CEST contrast following the RGD-CEST-liposomes injection.

The images obtained in vivo show that RGD-CEST-liposomes are able to target tumoral tissue. The higher % CEST contrast elevation is observed within the tumor and its surroundings in comparison with the whole brain.

1. Method of CEST imaging a subject comprising the steps of administering into the subject a diagnostic composition containing a CEST contrast agent, the CEST contrast agent being a composition comprising an encapsulating system ES encapsulating at least one shift agent, wherein the at least one shift agent is constituted of a monomeric chelate q≧2, or of a multimer of monomeric chelates q≧2, and wherein said chelate is free inside the encapsulating system and imaging said subject using a CEST based MRI procedure.

2. The method as claimed in claim 1, wherein the monomeric chelate is a q=2 chelate.

3. The method as claimed in claim 2, wherein the chelate is chosen from: PCTA, DO3A, DO3MA, AAZTA, HOPO and derivatives thereof.

4. The method as claimed in claim 1, wherein the chelate is a q=3 chelate.

5. The method as claimed in claim 4, wherein the q=3 chelate is chosen from HOPO, PC2A, BP2A and Tx.

6. The method as claimed in claim 1, wherein the multimer of monomeric chelates is a dimer, a trimer or a tetramer of a monomeric chelate q≧2.

7. The method as claimed in claim 1, wherein the metal of the shift agent is chosen from Dy3+, Tb3+, Tm3+,Yb3+, Eu3+ and Gd3+.

8. The method as claimed in claim 1, wherein the metal chelate is chosen from PCTA-Tm, PCTA-Dy, DO3A-Tm and DO3A-Dy.

9. The method as claimed in claim 1, wherein the encapsulating system is a liposome, a water/oil/water double emulsion, a water-in-oil emulsion or an inverse micelle.

10. The method as claimed in claim 1, wherein the encapsulating system is a nonspherical liposome.

11. The method as claimed in claim 1, wherein the encapsulating system also encapsulates a second monomeric q≧2 chelate which is different.

12. The method as claimed in claim 2, wherein the monomeric chelate is a q=2 chelate and wherein the encapsulating system also encapsulates a q=1 chelate.

13. The method as claimed in claim 11, wherein at least the second chelate is associated with the membrane.

14. The method as claimed in claim 1, wherein the encapsulating system also comprises at least one biovector for targeting a pathological region of interest.

15. The method as claimed in claim 3, wherein the chelate is chosen from PCTA, DO3A and AAZTA.

16. The method as claimed in claim 6, wherein the multimer of monomeric chelate is a dimer, a trimer or a tetramer of a monomeric chelate q=2.

17. The method as claimed in claim 14, wherein the biovector for targeting a pathological region of interest is an amino acid, a peptide, a polypeptide, a vitamin, a monosaccharide or polysaccharide, an antibody, a nucleic acid, a biovector targeting cell receptors, a pharmacophor, an angiogenesis-targeting biovector, an MMP-targeting biovector, a tyrosine-kinase-targeting peptide, an atheroma-plaque-targeting peptide or an amyloid-plaque-targeting biovector.
